(19) United States
(12) Patent Application Publication
(10) Pub. No.: US 2026/0308326 A1
(43) Pub. Date: Oct. 8, 2026

(54) Polyether Sulfone Copolymers and Articles Prepared Therefrom

(71) Applicant: Thermo Fisher Bioprocessing Inc., Waltham, MA (US)

(72) Inventors: Jonathan F. Hester, Hudson, WI (US); George W. Griesgraber, Eagan, MN (US); David Scott Hays, Waltham, MA (US); Ken Kawamoto, Waltham, MA (US); Austin Kruger, Waltham, MA (US); Lucas D. McIntosh, Minneapolis, MN (US)

(21) Appl. No.: 19/488,978

(22) PCT Filed: May 30, 2024
(86) PCT No.: PCT/IB2024/055281

§ 371 Date: Dec. 1, 2025

Related U.S. Application Data
(60) Provisional application No. 63/505,098, filed on May 31, 2023.

(51) Int. Cl. C08G 75/23; B01D 61/14; B01D 69/04; B01D 69/08; B01D 71/52; B01D 71/68

(52) U.S. Cl. CPC C08G 75/23; B01D 61/14; B01D 69/04; B01D 69/08; B01D 71/5221; B01D 71/68

(57) ABSTRACT

Polyether sulfone copolymers and porous polymeric articles containing the copolymers are provided. The copolymers are formed from a reaction mixture containing (a) a functional diphenyl-containing macromer having a grafted sidechain and (b) functional diphenylsulfone. The porous polymeric article is typically a membrane that is often either a flat sheet or a hollow fiber. The porous polymeric articles can be used, for example, to separate mixtures of biomaterials having different average sizes based on the average pore size of the porous polymeric articles. For example, biomaterials such as proteins, viruses, and cells can be separated based on size.

### BACKGROUND

Separation devices having a porous polymeric substrate have been used in a wide range of different industrial, pharmaceutical, and medical applications. Some of these separation devices function by separating a mixture of materials based on the average size of the materials in the mixture. These separation devices typically retain materials of a certain size such as those having a diameter larger than the diameter of the pores in the polymeric substrate while allowing passage of other materials having a diameter smaller than the diameter of the pores in the polymeric substrate.

Membranes formed from polyether sulfone (PES) have been used for separating biological materials such as proteins, viruses, and cells based on size. The pore size of PES membranes can be tuned during membrane casting to suit a particular filtration need. Additionally, hydrophilic homopolymers such as, for example, poly(vinyl pyrrolidone) (PVP), poly(oxazoline) (POx), and poly(ethylene glycol) (PEG) can be added to the membrane casting solution to render the membrane hydrophilic and therefore somewhat resistant to fouling by biological components, oils, surfactants, and other fluid components that tend to adhere to membrane surfaces. However, membranes cast with these hydrophilic homopolymers frequently are not uniformly hydrophilic and remain susceptible to fouling when contaminated compositions are passed through them. Additionally, because these hydrophilic homopolymers are typically water soluble, a fraction of them can be extracted from the membrane during use. Therefore, users typically perform a pre-flush of the membrane to lower the extractable content to a suitable level before filtering the compositions of interest.

### SUMMARY

Polyether sulfone copolymers and porous polymeric articles containing the copolymers are provided. The copolymers are formed from a reaction mixture containing (a) a functional diphenyl-containing macromer having a grafted sidechain and (b) functional diphenylsulfone. The porous polymeric article is typically a membrane that is often either a flat sheet or a hollow fiber. The porous polymeric articles can be used, for example, to separate mixtures of biomaterials having different average sizes based on the average pore size of the porous polymeric articles. For example, biomaterials such as proteins, viruses, and cells can be separated based on size.

In a first aspect, a copolymer is provided that comprises a plurality of repeat units joined by —O— groups. The plurality of repeat units includes (a) repeat units of Formula (I)

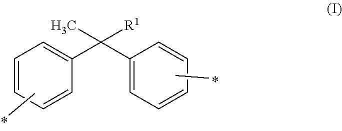

and repeat units of Formula (II).

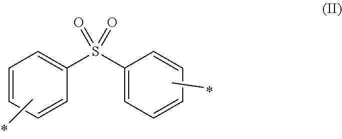

In Formula (I), the group R¹ comprises a plurality of repeat groups of formula —(CH₂)_{y}—X— where X is —O— or —NH— and where each y is an integer in a range of 1 to 4. More particularly, R¹ comprises a group of formula —[(CH₂)_{y}—X]ₙ— where n is an integer in a range of 3 to 1000. An asterisk (*) is an attachment site to the —O— group that joins two repeat units.

In a second aspect, a porous polymeric article is provided that comprises the copolymer described above in the first aspect. In most embodiments, the porous polymeric article is a membrane.

In a third aspect, a method of separating biomaterials based on size is provided. The method includes providing a porous polymeric article as described above in the second aspect and passing an aqueous mixture of biomaterials through the porous polymeric article, wherein the mixture of biomaterials comprises a plurality of biomaterials having different average sizes. The method further includes separating the mixture of biomaterials based on their average sizes, wherein a first biomaterial that is smaller than a second biomaterial permeates through the porous polymeric article at a faster rate.

As used herein, the terms “a”, “an”, “the”, and “at least one” are used interchangeably.

The term “and/or” means either or both. For example, “A and/or B” means A alone, B alone, or both A and B.

The term “alkylene” refers to a divalent group that is a radical of an alkane. The alkylene group can have 1 to 32 carbon atoms, 1 to 20 carbon atoms, 1 to 12 carbon atoms, 1 to 10 carbon atoms, 1 to 6 carbon atoms, or 1 to 4 carbon atoms. The alkylene can be linear, branched, cyclic, or a combination thereof. A linear alkylene has at least one carbon atom while a cyclic or branched alkylene has at least 3 carbon atoms.

The term “leaving group” refers to a group that can depart from a compound with a pair of electrons such as, for example, —F, —Cl, —Br, —I, CF₃SO₃—, and —SO₂—C₆H₄—CH₃.

The term “nucleophilic group” refers to a group that has an electron-rich atom that can donate a pair of electrons to form a covalent bond. As used herein, the nucleophilic group is often hydroxy (—OH) or —O—Si(R^{c})₃ where R^{c} is an alkyl or aryl.

The term “macromer” is used herein to refer to a reactive compound having a grafted polymeric group. More particularly, as used herein, the term macromer refers to a functional diphenyl-containing compound having (1) at least two functional groups that are leaving groups or nucleophilic groups and (2) a covalently attached (grafted) polymeric sidechain. The number of functional groups is usually equal to two. The macromer can be referred to as a polymer.

The term “monomer” is used herein to refer to a reactive compound that does not have a polymeric group. The monomer is typically a compound with at least two functional groups that are leaving groups or nucleophilic groups. The number of functional groups is usually equal to two.

The terms “polymer” and “polymeric material” are used interchangeably and refer to materials formed by reacting one or more monomers and/or macromers. The terms include homopolymers, copolymers, terpolymers, or the like. Likewise, the terms “polymerize” and “polymerizing” refer to the process of making a polymeric material that can be a homopolymer, copolymer, terpolymer, or the like. The term “copolymer” is used herein to refer to a polymer and polymeric materials derived from more than one type of monomer and/or macromer.

The terms “casting solution” and “polymer dope” are used interchangeably to refer to the composition used to form a porous membrane.

The term “membrane” refers to a porous article that is formed from a polymeric composition. The membrane can be in the form of a flat sheet or a hollow fiber. The membranes described herein are typically prepared using a phase separation process.

For any stated range, the endpoints are considered part of the range.

### BRIEF DESCRIPTION OF THE DRAWINGS

FIG. 1 illustrates a perspective view of a partial cross-section of a portion of an exemplary hollow fiber membrane.

FIG. 2 illustrates a cross-section view of an exemplary hollow fiber membrane.

FIG. 3 is a scanning electron micrograph of a cross section of a hollow fiber membrane of Example HFM1 at a magnification of 200 times.

FIG. 4 is a scanning electron micrograph of a cross section of a hollow fiber membrane of Example HFM1 at a magnification of 1,000 times.

FIG. 5 is a scanning electron micrograph of a lumen wall of a hollow fiber membrane of Example HFM1 at a magnification of 5,000 times.

FIG. 6 is a scanning electron micrograph of an outside wall of a hollow fiber membrane of Example HFM1 at a magnification of 5,000 times.

FIG. 7 is a scanning electron micrograph of a cross section of a comparative hollow fiber membrane of Comparative Example CHFM1 at a magnification of 200 times.

FIG. 8 is a scanning electron micrograph of a cross section of a comparative hollow fiber membrane of Comparative Example CHFM1 at a magnification of 1,000 times.

FIG. 9 is a scanning electron micrograph of a lumen wall of a comparative hollow fiber membrane of Comparative Example CHFM1 at a magnification of 5,000 times.

FIG. 10 is a scanning electron micrograph of an outside wall of a comparative hollow fiber membrane of Comparative CHFM1 at a magnification of 5,000 times.

FIG. 11 is a plot of flux of a 0.1 weight percent TWEEN-80 solution divided by flux of a buffer solution without TWEEN-80 as a function of volumetric throughput for membrane modules containing a hollow fiber membrane of Example HFM1 and Comparative Example CFM1.

### DETAILED DESCRIPTION

Copolymers and porous polymeric articles containing the copolymers are provided. The copolymers are formed from a reaction mixture containing (a) a functional diphenyl-containing macromer having a grafted polymeric sidechain and (b) functional diphenylsulfone. The porous polymeric article containing the copolymer is typically a membrane that is often either a flat sheet or a hollow fiber. The porous polymeric articles can be used, for example, to separate mixtures of biomaterials having different average sizes based on the average pore size of the porous polymeric articles. For example, biomaterials such as proteins, viruses, and cells can be separated based on size.

Advantageously, because the copolymers include macromeric units having covalently attached polymeric sidechains that are hydrophilic, the amount of pre-flushing that needs to be done to remove extractable hydrophilic polymers from a porous polymeric article prepared from these copolymers can be substantially reduced. Additionally, the grafted polymeric sidechains of the copolymer result in the formation of membranes with uniform hydrophilic surfaces. Consequently, these membranes tend to have greater resistance to fouling than those formed using hydrophilic homopolymers. Additionally, the copolymers with grafted polymeric sidechains are typically amphiphilic but not water soluble. Because these copolymers are typically not water soluble, they are usually less extractable than the previously used hydrophilic homopolymers.

Functional Diphenyl-Containing Macromers with Grafted Polymeric Sidechains

Functional diphenyl-containing macromers are provided with grafted polymeric sidechains. These macromers with grafted polymeric sidechains typically have at least two functional groups that can react with other monomers to form a copolymer. The functional groups can be either leaving groups or nucleophilic groups.

The functional diphenyl-containing macromers are of Formula (III)

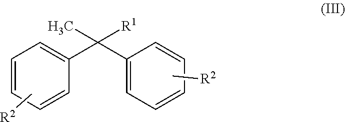

Group R¹ comprises a plurality of repeat groups of formula —(CH₂)_{y}—X— where X is —O— or —NH—. More particularly, R¹ comprises a group of formula —[(CH₂)_{y}—X]ₙ— where n is an integer in a range of 3 to 1000. Each y is an integer in a range of 1 to 4 such as in a range of 1 to 3, in a range of 2 to 3, or equal to 2. Each R² is a functional group that is independently a leaving group or a nucleophilic group. The compound of Formula (III) is usually of Formula (III-A).

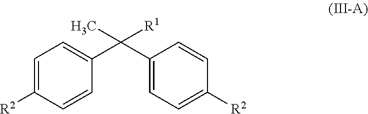

In many embodiments, group R¹ is a group of formula —R³—C(═O)—Z—[(CH₂)_{y}—X]ₙ—R⁴ wherein R³ is an alkylene, Z is —O— or —NH—, X is —O— or —NH—, and R⁴ is a terminal group. Variable y is an integer in a range of 1 to 4 and variable n is an integer in a range of 3 to 1000. Group X is often —O— and Z is either —O— or —NH—. That is, the compound of Formula (III) is of Formula (III-1)

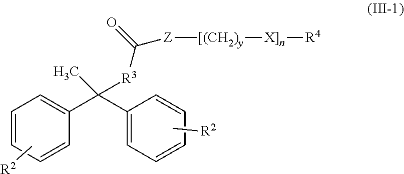

and the compound of Formula (III-A) is of Formula (III-A-1).

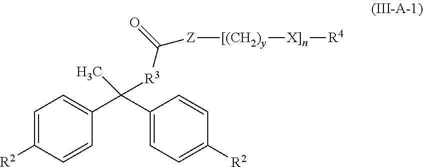

In many embodiments of compounds Formulas (III-1) and (III-A-1), X is —O— and the group —[(CH₂)—X]ₙ— is a poly(alkylene oxide) such as poly(butylene oxide), poly(propylene oxide), poly(ethylene oxide), or poly(methylene oxide) group.

The group n in Formulas (III-1) and (III-A-1) is an integer equal to at least 3 and is often in a range of 3 to 1000. That is, n is at least 3, at least 5, at least 10, at least 15, at least 20, at least 25, at least 30, at least 35, at least 40, at least 45, at least 50, at least 100, at least 200, at least 300, at least 400, or at least 500 and up to 1000, up to 900, up to 800, up to 700, up to 600, up to 500, up to 400, up to 300, up to 200, up to 100, up to 90, up to 80, up to 70, up to 60, up to 50, up to 40, up to 30, or up to 20. For example, variable n can be in a range of 3 to 500, 3 to 100, 5 to 100, or 5 to 50.

Group R³ in Formulas (III-1) and (III-A-1) is usually an alkylene. Although the alkylene can have any suitable number of carbon atoms, R³ is often ethylene. That is, group R¹ is often of formula —CH₂CH₂—C(═O)—Z—[(CH₂)_{y}—X]ₙ—R⁴.

Group R⁴ in Formula (III-A-1) is a terminal group. Although any suitable terminal group can be used, R⁴ is often an alkyl. The alkyl group often has 1 to 4 carbon atoms, 1 to 3, or 1 to 2 carbon atoms. Often, R⁴ is methyl.

Each group R² in Formulas (III), (III-A), and (III-A-1) is independently a leaving group or a nucleophilic group. Suitable leaving groups include, for example, —F, —Cl, —Br, —I, CF₃SO₃—, and —SO₂—C₆H₄—CH₃. Suitable nucleophilic groups include, for example, —OH or —O—Si(R⁶)₃ where each R⁶ group is an alkyl such as those having 1 to 4 carbon atoms. If desired, the nucleophilic group —O—Si(R⁶)₃ can be converted to a —OH group after formation of the compound of Formula (III) having the grafted polymeric sidechains. It is the group R² that is reactive (i.e., functional) when a polymeric material (i.e., copolymer) is formed that includes repeat units derived from the macromer of Formula (III).

Any suitable method can be used to form the compound of Formula (III-A). One example of such a method is shown in Reaction Scheme A.

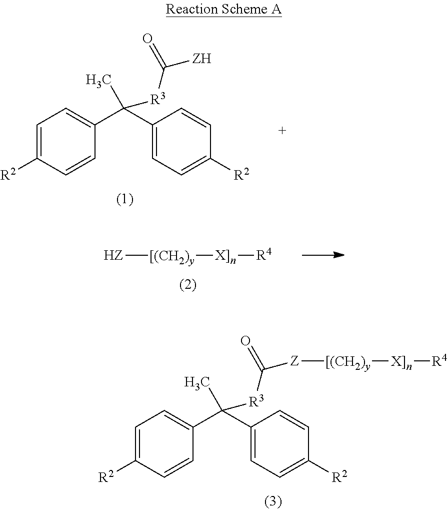

Compound (1) is often diphenolic acid where Z is equal to —O—, R³ is ethylene, and each R² is hydroxyl. Compound (2) is often a polyethylene glycol mono-amine (X is equal to —O— and Z is —NH—) or polyethylene glycol mono-alcohol (X is —O—, Z is —O—) where R⁴ is usually methyl. Compound (3) is the compound of Formula (III-A-1).

The functional diphenyl-containing macromers of Formula (III) (such as those of Formulas (III-1), (III-A) and (III-A-1)) typically have a weight average molecular weight (Mw) ranging from 350 to 50,000 Daltons. The Mw is often at least 350, at least 400, at least 500, at least 1000, at least 1500, at least 2000, at least 2500, at last 3000, at least 4000, at least 5000, at least 10,000, at least 15,000, or at least 20,000 Daltons and up to 50,000, up to 45,000, up to 40,000, up to 30,000, up to 25,000, up to 20,000, up to 15,000, up to 10,000, up to 5,000 Daltons, up to 2,000 Daltons, or up to 1,000 Daltons. For example, the range can be from 380 to 20,000, 500 to 10,000, 500 to 5000, 500 to 2000, 500 to 1000, 1000 to 10,000, 2500 to 10,000, 2000 to 10,000, 2000 to 5000, or 2500 to 5000 Daltons. The weight average molecular weight of the compounds of Formula (I) can be determined by Nuclear Magnetic Resonance (NMR) spectroscopy as described in the Examples below.

Copolymers Having Repeat Units Derived from Functional Diphenyl-Containing Macromers with Grafted Polymeric Sidechains

The functional diphenyl-containing macromers of Formula (III) having grafted polymeric sidechains of formula R¹ can be used to form copolymers. For ease of discussion, this copolymer is herein referred to as “POLY-1”. POLY-1 is an amphiphilic graft copolymer. POLY-1 is typically formed by reacting a first macromer of Formula (III) having two functional groups R²

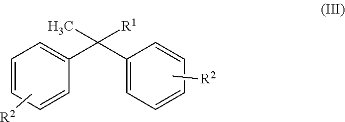

with a second monomer of Formula (IV).

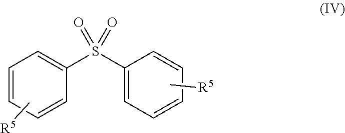

having two functional groups R⁵ that are either nucleophilic groups or leaving groups. In many embodiments, the first macromer of Formula (III) is of Formula (III-A) and the second monomer of Formula (IV) is of Formula (IV-A).

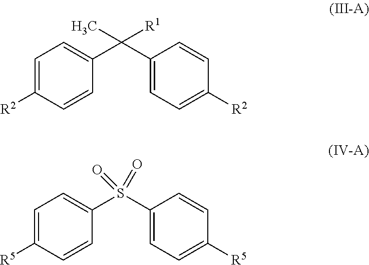

The reaction product is typically a copolymer (POLY-1) with a plurality of repeat units connected via an —O— group. The plurality of repeat units includes (a) repeat units of Formula (I)

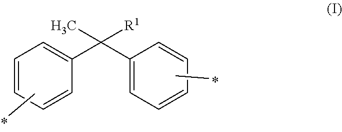

derived from the macromer of Formula (III) and repeat units of Formula (II)

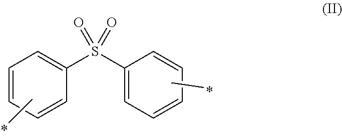

derived from the monomer of Formula (IV). Each asterisk (*) is an attachment site to an —O— group that joins repeat units. In many embodiments, the plurality of repeat units of Formula (I) are of Formula (I-A) and the plurality of repeat units of Formula (II) are of Formula (II-A).

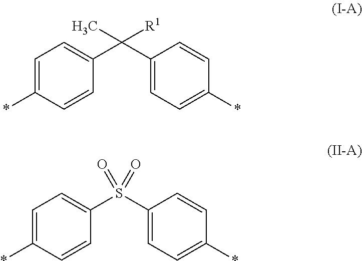

The repeat unit of Formula (I-A) is derived from the macromer of Formula (III-A) and the repeat unit of Formula (II-A) is derived from the monomer of Formula (IV-A).

If R² in the first macromer of Formula (III) is a leaving group (L) as shown in Formula (III-B),

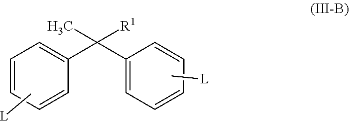

then at least some of the second monomer of Formula (IV) has two hydroxy nucleophilic groups as shown in Formula (IV-1) or two group of formula —O—Si(R^{c})₃ where each R¹ is an alkyl or aryl as shown in Formula (IV-2).

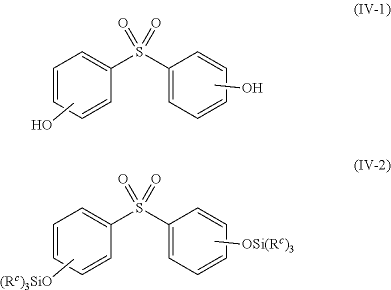

In most embodiments, the second monomer with nucleophilic groups is of Formula (IV-1). Often, the first macromer is of Formula (III-B-1)

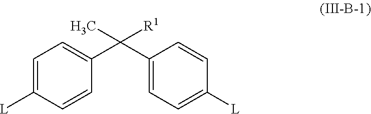

where L is —F, —Cl, —Br, —I, CF₃SO₃—, or —SO₂—C₆H₄—CH₃ and the second monomer is of Formula (IV-A-1).

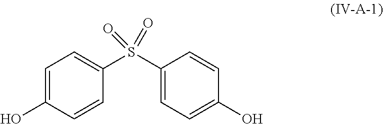 [SMILES: O=S(=O)(c1ccc(O)cc1)c1ccc(O)cc1]

If R² in the first macromer of Formula (III) is a nucleophilic group such as hydroxy, however, as shown in Formula (III-C),

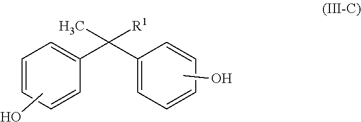

then at least some of the second monomer of Formula (IV) has two leaving groups as shown in Formula (IV-3).

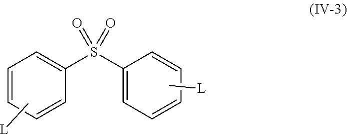

In most embodiments, the first macromer is of Formula (III-C-1)

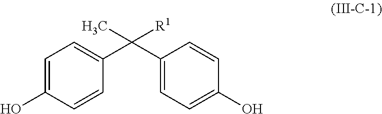

and the second monomer is of Formula (IV-A-2)

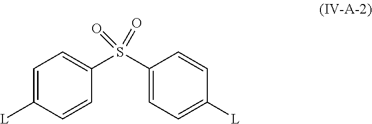

where each L is typically —F, —Cl, —Br, —I, CF₃SO₃—, or —SO₂—C₆H₄—CH₃.

In many embodiments, the molar ratio of the first macromer of Formula (III) to the second monomer of Formula (IV) is less than 1. To provide a copolymer with molar ratios less than 1, a mixture of second monomers of Formula (IV) is used with some of the second monomers having R⁵ nucleophilic groups and other second monomers having R⁵ leaving groups. Thus, the second monomer is often a mixture of monomers of Formula (IV-1) and/or Formula (IV-2) plus monomers of Formula (IV-3). In many embodiments, the second monomer is a mixture of monomers of Formula (IV-1) and Formula (IV-3). The first macromer and second monomers are combined such that the total moles of nucleophilic groups are equal to or approximately equal to the moles of leaving groups.

In many embodiments, the second monomer is of Formula (IV-A-1), Formula (IV-A-2), or a mixture thereof.

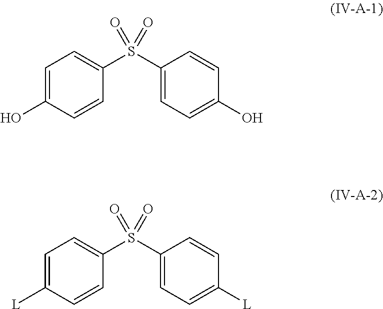

The monomer of Formula (IV-A-1) is within the scope of Formulas (IV-A) and (IV-1) above while the monomer of Formula (IV-A-2) is within the scope of Formula (IV-A) and (IV-3) above. In many embodiments, L is —Cl or —F.

POLY-1 can include additional optional repeat units in addition to those of Formulas (I) and (II) described above. These optional repeat units include the following third repeat units of Formula (V-A), Formula (V-B), Formula (V-C), Formula (V-D), Formula (V-E), Formula (V-F) or a combination thereof.

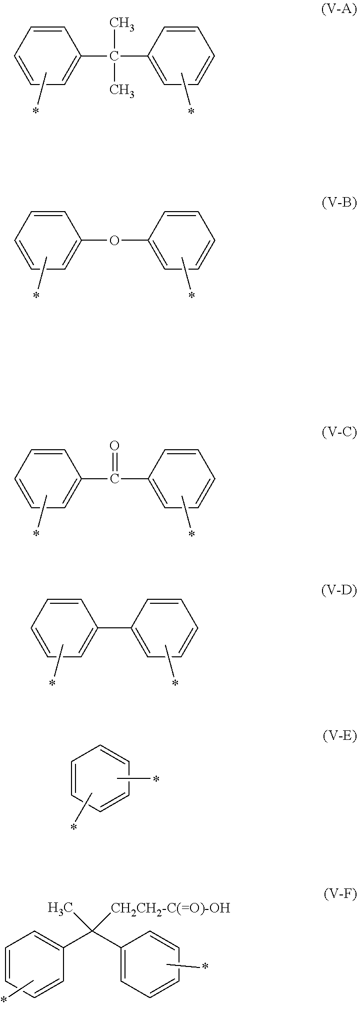

wherein an asterisk (*) is the attachment site to an —O— group that joins repeat units. In many embodiments these repeat units are derived from monomers such as, for example, those of Formula (VI-A) to (VI-F) respectively or isomers thereof. The monomeric units of Formula (VI-F) can result, for example, from hydrolysis of some of the monomer of Formula (III). In these formulas, the group R⁸ is either a nucleophilic group or a leaving group as described above. In many embodiments, R⁸ is hydroxy.

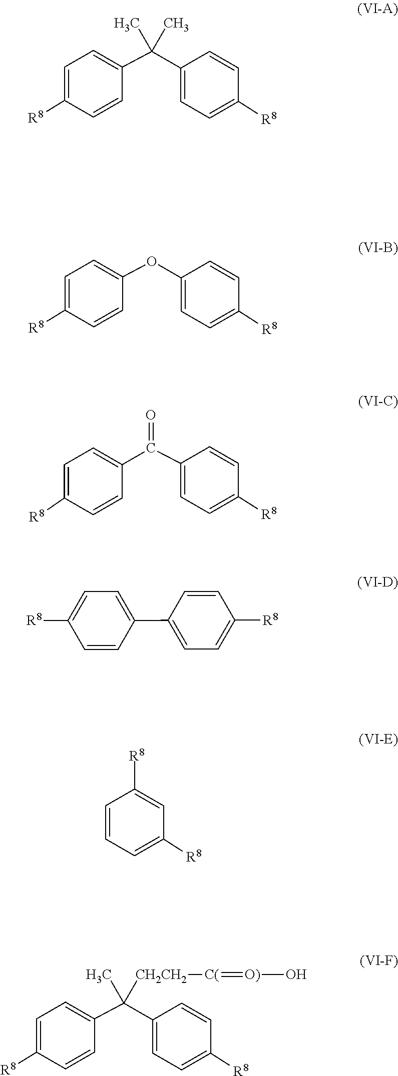

While any amount of the first macromer of Formula (III) can be used to form POLY-1, the amount often needs to be controlled if the resulting copolymer POLY-1 is used to prepare a membrane such as those described below. For example, if the membrane is prepared using a phase separation process, the amount of first macromer of Formula (III) typically needs to be controlled so that POLY-1 is amphiphilic but not water soluble. If the amount of the first macromer of Formula (III) is too low, a membrane formed from the copolymer tends not to have sufficient antifouling characteristics and biomaterials such as proteins can undesirably stick to its surface. Increasing the amount of the first macromer of Formula (III) used to form the graft copolymer POLY-1 tends to increase the amount of POLY-1 on the membrane surface during solvent induced phase separation (SIPS) casting and tends to provide the desired membrane surface properties (e.g., water wettability and fouling resistance) at lower concentrations of POLY-1 in the casting solution. If the content of the first macromer of Formula (III) is too great, however, POLY-1 may be water soluble. Water solubility of POLY-1 is generally undesirable because this can lead to loss of the copolymer into the precipitation bath during membrane casting and/or to higher water extractability of the graft copolymer from the finished membrane. Thus, it is often desirable to prepare POLY-1 with the maximum amount of the macromer of Formula (III) that can be incorporated while not rendering POLY-1 water soluble. This maximum amount depends on the hydrophilicity of the grafted polymeric sidechains, which in turn depends on the composition of the grafted polymeric sidechains.

The amphiphilic graft copolymer POLY-1 is often prepared from a polymerizable composition that contains 10 to 60 weight percent of a first macromer of Formula (III) based on a total weight of the polymerizable material (i.e., macromers and monomers). The amount of the first macromer is often at least 10, at least 15, at least 20, at least 25, or at least 30 weight percent and up to 60, up to 55, up to 50, up to 45, up to 40, up to 35, or up to 30 weight percent based on the total weight of polymerizable composition. In some embodiments, the amount of the first macromer of Formula (III) ranges from 10 to 55, 10 to 50, 10 to 45, 10 to 40, 10 to 30, 15 to 60, 15 to 55, 15 to 50, 15 to 45, 15 to 40, 20 to 60, 20 to 55, 20 to 50, 20 to 45, 20 to 40, 20 to 35, or 20 to 30 weight percent based on the total weight of polymerizable composition.

In addition to the macromer of Formula (III), the polymerizable composition used to form POLY-1 can contain 40 to 90 weight percent of a second monomer of Formula (IV). The amount of the monomer of Formula (IV) can be at least 40, at least 45, at least 50, at least 55, at least 60, at least 65, or at least 70 and up to 90, up to 85, up to 80, up to 75, up to 70, up to 65, or up to 60 weight percent based on a total weight of polymerizable composition. In some embodiments, the amount of the second monomer of Formula (IV) ranges from 45 to 90, 50 to 90, 55 to 90, 60 to 90, 70 to 90, 40 to 85, 45 to 85, 50 to 85, 55 to 85, 60 to 85, 40 to 80, 45 to 80, 50 to 80, 55 to 80, 60 to 80, 65 to 80, or 70 to 80 weight percent based on the total weight of polymerizable composition.

In addition to the macromer of Formula (III) and the monomer of Formula (IV), the polymerizable composition used to form POLY-1 can optionally contain 0 to 50 weight percent of a third monomer of Formula (V-A), (V-B), (V-C), (V-D), (V-E), (V-F), or a mixture thereof. The amount of this third monomer, if present, can be at least 1, at least 2, at least at least 5, at least 10, or at least 15 and up to 50, up to 40, up to 30, up to 25, up to 20, up to 15, up to 10, or up to 5 weight percent based on the total weight of polymerizable composition.

POLY-1 typically has a weight average molecular weight (Mw) ranging from 10,000 to 250,000 Daltons. The weight average molecular weight is typically at least 10,000, at least 15,000, at least 20,000 at least 25,000, at least 30,000, at least 35,000, at least 40,000, at least 45,000, at least 50,000, at least 75,000, or at least 100,000 Daltons and up to 250,000, up to 225,000, up to 200,000, up to 175,000, up to 150,000, up to 125,000, up to 100,000, up to 80,000, up to 60,000, up to 50,000, up to 40,000, up to 35,000, or up to 30,000 Daltons. In some embodiments, the range is from 20,000 to 200,000, 25,000 to 100,000, from 25,000 to 80,000, from 25,000 to 60,000, from 25,000 to 50,000, or from 25,000 to 40,000 Daltons. The weight average molecular weight can be determined by gel permeation chromatography (GPC) as described in the Examples below.

### Porous Polymeric Articles

Porous polymeric articles are provided that contain an amphiphilic grafted copolymer POLY-1 having repeat units derived from the functional diphenyl-containing macromers with grafted polymeric sidechains as described above. Although the porous polymeric article can be in any form, it typically is a membrane. The membrane can be, for example, in the form of a flat sheet or a hollow fiber. The membranes are typically formed by a phase separation process.

In many embodiments, the phase separation process used to form the membrane is a solvent induced phase separation (SIPS) process and the resulting membrane can be referred to as a “SIPS membrane”. SIPS membranes are sometimes referred to by other names such as, for example, “DIPS membranes” formed by diffusion induced phase separation, “NIPS membranes” formed by non-solvent induced phase separation, or as “phase inversion membranes”. These processes are all referred to herein as SIPS processes and the products as SIPS membranes.

In a SIPS process, the polymeric material is combined with other solution components such as a suitable solvent for the polymeric material to prepare a substantially homogeneous solution that is referred to interchangeably as a “casting solution” or as a “polymer dope”. The casting solution is formed into either a flat sheet using a coating process or a hollow tube using a spinning process. The flat sheet or hollow fiber is then immersed in a “quench solution” containing a non-solvent for the polymeric material. Exchange of the solvent in the casting solution with the non-solvent in the quench solution results in phase separation of the polymer solution yielding a solid, porous flat sheet or hollow fiber. In some embodiments, the flat sheet or hollow fiber can be exposed to a humid atmosphere or steam prior to immersion into the quench solution to begin the phase separation process.

The casting solution used to form the SIPS membrane typically includes a mixture of polymeric materials and a water-miscible organic solvent. The mixture of polymeric materials includes POLY-1 as described above having grafted polymeric sidechains, a second polymer POLY-2 that is typically an aromatic polyether sulfone lacking grafted polymeric sidechains, and an optional third polymer POLY-3 that is typically a hydrophilic pore former. In addition to the polymeric materials, the casting solution also contains a water-miscible solvent that can dissolve POLY-1, POLY-2, and optional POLY-3. Still further, the casting solution can optionally contain water.

The casting solution contains POLY-1, which is the copolymer described above that comprises (1) repeat units of Formula (I)

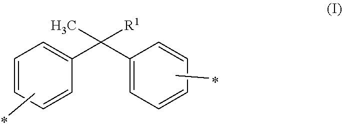

that is derived from a difunctional diphenyl-containing macromer with a grafted polymeric sidechain R¹ as well as (2) repeat units of Formula (II)

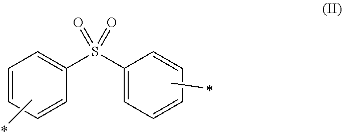

that is derived from a difunctional diphenylsulfone without grafted polymeric sidechains. The casting solution typically contains 1 to 20 weight percent of POLY-1 based on a total weight of the casting solution. The amount can be at least 1, at least 1.5, at least 2, at least 2.5, at least 3, at least 4, or at least 5 weight percent and up to 20, up to 18, up to 15, up to 12, up to 10, up to 8, or up to 5 weight percent based on the total weight of the casting solution. For example, the amount of POLY-1 can be in a range of 1.5 to 20, 1.5 to 15, 2 to 20, 2 to 15, 2 to 10, or 2 to 5 weight percent based on the total weight of the casting solution. The various repeat units are connected by —O— linkages.

In addition to POLY-1, the casting solution includes a second polymer that can be referred to as POLY-2. POLY-2 is an aromatic polyether sulfone having repeat units of Formula (II)

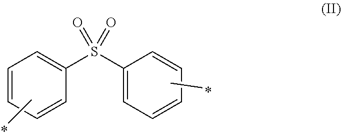

wherein an asterisk (*) is the attachment site to an —O— group that joins two repeat units. This second polymer can be a homopolymer or copolymer. If POLY-2 is a copolymer, it is free of repeat units of Formula (I) and is made from a polymerizable mixture that is free of the macromer of Formula (III). POLY-2 lacks the grafted polymeric sidechains that are included in POLY-1.

In some embodiments of POLY-2, the repeat units of Formula (II) are combined with one or more optional repeat units of Formula (V-A), Formula (V-B), Formula (V-C), Formula (V-D), Formula (V-E), Formula (V-F) or a combination thereof as described above as optional repeat units that can be included in POLY-1. Adjacent repeat units are typically connected by an —O— group. These optional repeat units typically are no greater than 50 mole percent of all the repeat units in POLY-2. That is, they can be present in an amount of 0 to 50 mole percent based on all the repeat units included in POLY-2. For example, these optional repeat units can be up to 50, up to 45, up to 40, up to 35, up to 30, up to 25, up to 20, up to 15, up to 10, or up to 5 mole percent of all the repeat units in POLY-2.

POLY-2 typically has a weight average molecular weight (Mw) in a range of 30,000 to 150,000 Daltons (30 to 150 kDa). For example, Mw is often at least 30, at least 40, at least 50, at least 60, or at least 70 and up to 150, up to 140, up to 130, up to 120, up to 100, up to 90, up to 80, up to 70, up to 60, or up to 50 kDa. The range can be, for example, from 30 to 120, 50 to 120, or 60 to 100 kDa. The weight average molecular weight can be determined by gel permeation chromatography (GPC) as described in the Examples below.

The amount of POLY-2 in the casting solution is typically in a range of 10 to 40 weight percent based on the total weight of the casting solution. The amount can be at least 10, at least 12, at least 15, or at least 20 weight percent and up to 40, up to 35, up to 30, up to 25, up to 20, or up to 15 weight percent based on the total weight of the casting solution. For example, the amount of POLY-2 can be in a range of 10 to 35, 10 to 30, 10 to 25, 12 to 40, 12 to 35, 12 to 30, 12 to 25, 15 to 40, 15 to 35, 15 to 30, 15 to 25, 20 to 40, 20 to 35, or 20 to 30 weight percent based on the total weight of the casting solution. The amount of POLY-2 in the casting solution can impact the pore size of the resulting membrane formed from the casting solution. For example, an increase in the amount of POLY-2 tends to decrease the average pore size of the membrane. Varying the amount of POLY-2 can be used to alter the size of materials that can permeate though the membrane and/or the speed of permeation through the membrane.

An optional third polymeric material, which is referred to as POLY-3, is often included in the casting solution in addition to POLY-1 and POLY-2. This third polymer is typically selected to be a hydrophilic polymer that has greater water solubility than either POLY-1 or POLY-2. When phase separation occurs in the process of making the membrane, the third polymer (POLY-3) typically does not undergo phase separation. That is, POLY-3 remains in solution unlike both POLY-1 and POLY-2. Suitable POLY-3 hydrophilic polymers include, for example, poly(2-vinylpyrrolidone), polyethylene glycol, polyoxazoline, polyvinyl alcohol, polyglycol monoester, carboxylmethylcellulose, a polysorbitate such as polyoxyethylene sorbitan monooleate, carboxymethylcellulose polyacrylic acid, polyacrylamide, a copolymer thereof, or a blend thereof. In many embodiments, POLY-3 is a polyethylene glycol or a polymeric mixture that includes polyethylene glycol.

POLY-3 can have any desired molecular weight and may comprise a mixture of polymers of multiple molecular weights. In some embodiments the amount of one or more components of POLY-3 in the membrane is desired to be low to reduce its extractability. In such cases the weight average molecular weight of that component of POLY-3 is often less than 1000 Daltons. For example, the weight average molecular weight may be up to 750 Daltons such as in a range of 100 to 750, 100 to 500, 100 to 400, 200 to 750, 200 to 500, or 200 to 400 Daltons. In other embodiments the molecular weight of one or more components of POLY-3 in the membrane is selected to be higher to impart greater fouling resistance or to tune the porosity of the membrane. In such cases the weight average molecular weight of that component of POLY-3 is often greater than 1000 Daltons. For example, the weight average molecular weight of that component of POLY-3 can be up to 750,000 Daltons such as in a range of 1,100 to 750,000 Daltons, 1,100 to 500,000 Daltons, 1,100 to 400,000 Daltons, 2,000 to 500,000 Daltons, or 20,000 to 50,000 Daltons. The weight average molecular weight can be determined by gel permeation chromatography (GPC) as described in the Examples below.

The amount of POLY-3 in the casting solution is typically in a range of 0 to 65 weight percent based on a total weight of the casting solution. The amount can be at least 5, at least 10, at least 15, at least 20, at least 25, at least 30, at least 35, or at least 40 weight percent and up to 65, up to 60, up to 55, up to 50, up to 45, up to 40, or up to 35 weight percent based on the total weight of the casting solution. For example, the amount can be in a range of 1 to 65, 1 to 60, 5 to 60, 10 to 60, 10 to 50, 10 to 40, 10 to 30, 20 to 60, 20 to 50, 20 to 40, 30 to 30 to 60, 30 to 50, or 30 to 40 weight percent based on the total weight of the casting solution.

In addition to POLY-1, POLY-2, and POLY-3, the casting solution can include a water-miscible organic solvent. Water-miscible organic solvents that can be used include, for example, glycol, glycerol, butyrolactone, s-caprolactam, N-methylpyrrolidone, dimethyl sulfoxide, dimethyl acetamide, dimethyl formamide, and combinations thereof. In some embodiments, the water-miscible organic solvent includes N-methylpyrrolidone because it usually is a good solvent for both POLY-1 and POLY-2.

Any suitable amount of the water-miscible organic solvent can be included in the casting solution. The amount can be, for example, in a range of 20 to 70 weight percent based on a total weight of the casting solution. The amount can be at least 20, at least 25, at least 30, at least 35, at least 40, or at least 45 weight percent and up to 70, up to 65, up to 60, up to 55, up to 50, up to 45, or up to 40 weight percent based on the total weight of the casting solution. For example, the amount can be in a range of 20 to 65, 20 to 60, 20 to 55, 20 to 50, 20 to 45, 20 to 40, 25 to 65, 25 to 60, 25 to 55, 25 to 50, 25 to 45, 25 to 40, 30 to 60, 30 to 55, 30 to 50, 30 to 45, or 30 to 40 weight percent based on the total weight of the casting solution.

The casting solution can optionally include water. For example, the casting solution can contain 0 to 10 weight percent water. If present, the amount can be at least 1, at least 2, at least 3, at least 4, or at least 5 and up to 10, up to 8, up to 6, up to 5, or up to 4 weight percent based on the total weight of the casting solution. The amount can be, for example, in a range of 1 to 8, 1 to 6, 1 to 5, 2 to 10, 2 to 8, 2 to 6, 2 to 5, 3 to 10, 3 to 8, 3 to 6, or 3 to 5 weight percent based on the total weight of the casting solution.

In some embodiments, the casting solution contains 1 to 20 weight percent POLY-1, 10 to 40 weight percent POLY-2, 0 to 65 weight percent POLY-3, 20 to 70 weight percent water-miscible organic solvent, and 0 to 10 weight percent water based on a total weight of the casting solution. In some examples, the casting solution contains 1.5 to 15 weight percent POLY-1, 12 to 30 weight percent POLY-2, 20 to 50 weight percent POLY-3, 25 to 55 weight percent water-miscible organic solvent, and 2 to 6 weight percent water. In other examples, the casting solution contains 2 to 10 weight percent POLY-1, 15 to 25 weight percent POLY-2, 30 to 40 weight percent POLY-3, 30 to 40 weight percent water-miscible organic solvent, and 3 to 5 weight percent water. In most embodiments, the casting solution is transparent and macroscopically homogeneous.

The membrane that is formed from the casting solution can be either in the form of a sheet or a hollow fiber membrane. Both types of membranes can be formed from the casting solution. After forming either a sheet or hollow fiber, the sheet or the hollow fiber is immersed in a quenching bath that results in the precipitation of the membrane. If desired, the membrane can optionally be transferred to a second bath such as a water bath to extract additional materials from the membrane.

In many embodiments, the precipitation bath includes water and optionally can further include water-miscible organic solvents such as those listed above for use in the casting solution. For example, the precipitation bath often contains 50 to 100 weight percent water and 0 to 50 weight percent of a water-miscible organic solvent. In some embodiments, the precipitation bath includes 55 to 100 weight percent water and 0 to 45 weight percent water-miscible organic solvent or 60 to 100 weight percent water and 0 to 40 weight percent water-miscible organic solvent. The water-miscible solvent is often selected to be N-methylpyrrolidone.

While any suitable method can be used to form a membrane sheet, the casting solution is often heated to an elevated temperature such as, for example, in a range of 40 to 80 degrees Celsius. The casting solution can be coated onto a support that is also heated in a range of 40 to 80 degrees Celsius. The film can be prepared as a coating using, for example, a notch bar with a suitable gap height, such as in a range of 25.4 to 508 micrometers. The notch bar is often heated in a range of 40 to 80 degrees Celsius before spreading the casting solution out on the surface of the support. Immediately after forming a film of the casting solution on the support, the coated support is typically immersed into the precipitation bath that is often heated in a range of 40 to 80 degrees Celsius. If desired, the membrane optionally can be further extracted in a second bath that contains water. The membrane can be dried in an oven after formation. The temperature of the oven can be, for example, in a range of 40 to 80 degrees Celsius.

In some embodiments, the membrane is in the form of a sheet. The sheet typically has a thickness in a range of 30 to 250 micrometers. For example, the thickness can be at least 30, at least 40, at least 50, at least 60, at least 80, or at least 100 and up to 250, up to 225, up to 200, up to 175, up to 150, up to 125, up to 120, up to 110, or up to 100 micrometers. The range can be, for example, from 30 to 200, 30 to 150, 50 to 150, 50 to 125, 50 to 110, or 50 to 100 micrometers.

In many embodiments, the membrane is in the form of a hollow fiber. The hollow fiber can be formed by extruding the casting solution through a coaxial spinneret die. This coaxial spinneret die typically has an annular gap as well as a central inner channel that is arranged coaxially to the annular gap. The annular gap is separated from the central inner channel by a needle. The casting solution is introduced into the annular gap while a bore liquid is introduced into the inner chamber. The bore liquid stabilizes the lumen of the hollow fiber membrane as it is formed.

The spinneret die is selected depending on the desired dimensions of the hollow fiber membranes. While any suitable spinneret die can be used, the outer diameter of the annulus is often in a range of 300 to 1000 micrometers. The annulus outer diameter can be, for example, at least 300, at least 400, at least 500 and up to 1000, up to 800, or up to 600 micrometers. The inner diameter of the annulus, which is also the outer diameter of the needle, is often in a range of 190 to 980 micrometers. The annulus inner diameter can be, for example, at least 190, at least 200, at least 300, or at least 500 and up to 980, up to 900, up to 800, up to 600, or up to 500 micrometers. The inner diameter of the needle is often in a range of 40 to 830 micrometers. The needle inner diameter can be, for example, at least 40, at least 50, at least 75, at least 100, at least 150, at least 200 at least 250, at least 300, at least 350, at least 400 and up to 830, up to 800, up to 750, up to 700, up to 650, up to 600, up to 550, or up to 500 micrometers.

One exemplary spinneret die has an annulus outer diameter of 1000 micrometers, a needle outer diameter of 980 micrometers, an annular gap of 10 micrometers, a needle inner diameter of 830 micrometers, and a needle thickness of 75 micrometers. Another exemplary spinneret die has an annulus outer diameter of 500 micrometers, a needle outer diameter of 300 micrometers, an annular gap of 100 micrometers, a needle inner diameter of 150 micrometers, and a needle thickness of 75 micrometers. Yet another exemplary spinneret die has an annulus outer diameter of 410 micrometers, a needle outer diameter of 300 micrometers, an annular gap of 55 micrometers, a needle inner diameter of 150 micrometers, and a needle thickness of 75 micrometers. Still another exemplary spinneret die has an annulus outer diameter of 300 micrometers, a needle outer diameter of 190 micrometers, an annular gap of 55 micrometers, a needle inner diameter of 40 micrometers, and a needle thickness of 75 micrometers.

The bore liquid composition typically includes a water-miscible organic solvent, water, and optionally a hydrophilic polymer such as those listed above for POLY-3. Any suitable water-miscible organic solvent can be used such as those described above for use in the casting solution. In some embodiments, the water-miscible organic solvent used in the bore liquid includes N-methylpyrrolidone and any optional hydrophilic polymer used in the bore liquid includes polyethylene glycol.

The amount of the water-miscible organic solvent in the bore liquid composition is often in a range of 30 to 95 weight percent based on a total weight of the bore liquid. The amount can be at least 30, at least 35, at least 40, at least 45, or at least 50 weight percent and up to 95, up to 90, up to 85, up to 80, up to 75, up to 70, up to 65, up to 60, up to 55, or up to 50 weight percent. For example, the amount can be in a range of 30 to 90, 30 to 80, 30 to 75, 30 to 70, 30 to 60, 30 to 50, 40 to 90, 40 to 80, 40 to 75, 40 to 70, 40 to 60, or 40 to 50 weight percent.

The amount of water in the bore liquid composition is often in a range of 1 to 55 weight percent based on a total weight of the bore liquid. The amount can be at least 1.5, at least 2, at least 2.5, at least 3, at least 4, at least 5, at least 10 weight percent and up to 55, up to 50, up to 45, up to 40, up to 35, up to 30, up to 25, up to 20, up to 15, or up to 10 weight percent. For example, the amount can be in a range of 1 to 50, 1 to 40, 1 to 30, 1 to 20, 1 to 15, 1 to 10, 2 to 50, 2 to 40, 2 to 30, 2 to 20, 2 to 15, 2 to 10, 5 to 50, 5 to 40, 5 to 30, 5 to 20, or 5 to 15 weight percent.

The amount of the optional hydrophilic polymer in the bore liquid composition is often in a range of 0 to 60 weight percent based on the total weight of the bore liquid. The amount can be 0, at least 1, at least 5, at least 10, at least 15, at least 20, at least 25, at least 30, at least 35, at least 40 weight percent and up to 60, up to 55, up to 50, up to 45, up to 40, up to 35, or up to 30 weight percent. For example, the amount can be in a range of 0 to 50, 10 to 60, 10 to 55, 10 to 50, 10 to 45, 10 to 40, 20 to 60, 20 to 55, 20 to 50, 20 to 45, 20 to 40, 30 to 60, 30 to 55, 30 to 50, 30 to 45, 40 to 60, or 40 to 50 weight percent.

Overall, the bore liquid composition contains 30 to 95 weight percent water-miscible organic solvent, 1 to 55 weight percent water, and 0 to 60 weight percent of the optional hydrophilic polymer. In some examples, the bore liquid composition contains 35 to 80 weight percent water-miscible organic solvent, 2 to 40 weight percent water, and 20 to 55 weight percent hydrophilic polymer. In other examples, the bore liquid composition contains 35 to 75 weight percent water-miscible organic solvent, 2 to 20 weight percent water, and 30 to 55 weight percent hydrophilic polymer. In still other examples, the bore liquid composition contains 40 to 50 weight percent water-miscible organic solvent, 5 to 15 weight percent water, and 40 to 50 weight percent hydrophilic polymer. Typically, the bore liquid is transparent and macroscopically homogenous.

As noted above, the bore liquid is introduced into the inner chamber while the casting solution is introduced into the annular gap of the coaxial spinneret die. In some embodiments, each of the casting solution and the bore liquid is passed through a filter to remove any particulate materials prior to introduction into the annular gap and the inner chamber, respectively. Both fluid streams are often heated prior to introduction into the die. For example, both fluid streams can be heated in a range of 30 to 80 degrees Celsius. The flow rate of the bore liquid and the flow rate of the casting solution each usually can be independently controlled.

After leaving the coaxial spinneret die and before entering the precipitation bath, the extruded product optionally passes through a climate-controlled zone with defined climatic conditions. The climate-controlled zone is often in the form of an encapsulated chamber. The extruded product often has a retention time no greater than 10 seconds within the climate-controlled zone having a relative humidity of 20 to 95 percent and a temperature of 25 to 75 degrees Celsius. The relative humidity is often at least 40, at least 50, at least 55, at least 60, at least 65, at least 70, or at least 75 percent and up to 90, up to 85, up to 80, up to 75, or up to 70 percent. The temperature is often at least 25, at least 30, at least 35, at least 40, at least 45, or at least 50 degrees Celsius and up to 90, up to 85, up to 80, or up to 75 degrees Celsius. For example, the climate-controlled zone contains air with a relative humidity of 75 to 90% and a temperature of 30 to 50° C., a relative humidity of 60 to 75% and a temperature of 50 to 70° C., or a relative humidity of 75 to 90% and a temperature of 50 to 70° C.

The retention time within the climate-controlled zone is often at least 0.5, at least 1, at least 2, or at least 3 and up to 10, up to 8, up to 6, or up to 5 seconds. To establish stable conditions in the climate-controlled zone, the air often flows through the climate-controlled zone with a velocity of less than 0.5 m/s and preferably with a velocity in the range from 0.15 to 0.35 m/s.

As the extruded product is directed through the climate-controlled zone set to the climatic conditions, pre-coagulation may occur on the outside of the extruded product by absorption of water vapor acting as the non-solvent, before entering the precipitation bath.

After passing through the climate-controlled zone, the extruded product is directed into the precipitation bath that is in a range of 50 to 80 degrees Celsius to complete the formation of the hollow fiber membrane structure. The composition of the precipitation bath can be the same as described above to prepare membranes in the form of sheets. In the precipitation bath, the membrane structure is formed by precipitation (e.g., coagulation) and then stabilized. Extraction of the water-miscible solvents and the hydrophilic polymer occurs at the same time. That is, water, the water-miscible organic solvent, and the optional hydrophilic POLY-3 can be extracted. The pores of the membrane are formed within the precipitation bath due to the phase separation and extraction processes that occur.

After formation and extraction within the precipitation bath, the hollow fiber membrane can be processed using conventional methods. For example, the hollow fiber membrane optionally can be further treated by pouring deionized water through the lumen and extracted in water at an elevated temperature such as, for example, 70 to 100 degrees Celsius for several hours.

After drying either at room temperature or elevated temperatures, the hollow fiber membrane can be wound onto a coil or formed directly into bundles with a suitable fiber count and length. Before production of the bundles, supplementary threads in the form of multifilament yarns can be added to the hollow fiber membranes to ensure a spacing of the hollow fiber membranes relative to one another for better flow around the individual hollow fiber membranes in the bundle.

The membrane typically contains 1 to 50 weight percent POLY-1 based on a total weight of the membrane. For example, the membrane can contain at least 1, at least 2, at least 3, at least 5, at least 10, at least 15, at least 20, or at least 25 and up to 50, up to 45, up to 40, up to 35, up to 30, up to 25, or up to 20 weight percent POLY-1. The amount can be in a range of 2 to 50, 2 to 40, 3 to 30, 5 to 30, 5 to 25 or 5 to 20 weight percent based on a total weight of the membrane.

In addition to POLY-1, the membrane typically contains 50 to 99 weight percent POLY-2 based on a total weight of the membrane. The amount of POLY-2 can be at least 50, at least 55, at least 60, at least 65, at least 70, at least 75, or at least 80 and up to 99, up to 98, up to 97, up to 95, up to 90, up to 85, up to 80, up to 75 weight percent based on a total weight of the membrane. The amount of POLY-2 can be in a range of 50 to 98, 60 to 98, 70 to 97, 70 to 95, 75 to 95, or 80 to 95 weight percent based on a total weight of the membrane.

The membrane may optionally contain POLY-3, the water-miscible organic solvent, and/or water. The amount of POLY-3 is typically in a range of 0 to 20 weight percent based on the total weight of the membrane. For example, the amount of POLY-3 can be at least 0.1, at least 0.5, at least 1, at least 2, or at least 5 and up to 20, up to 15, up to 10, up to 8, or up to 5 weight percent. The amount of water-miscible organic solvent is typically in a range of 0 to 1 weight percent based on the total weight of the membrane. For example, the amount of the water-miscible organic solvent can be at least 0.05, at least 0.1, at least 0.2, at least 0.3, or at least 0.5 and up to 1, up to 0.8, up to 0.6, up to 0.5, up to 0.3, or up to 0.1 weight percent.

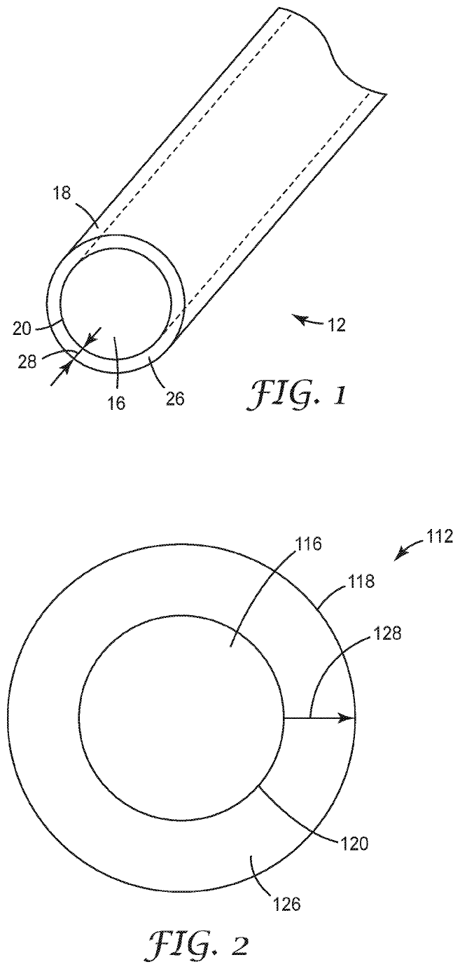

FIG. 1 illustrates a perspective view of a partial cross-section of a portion of an exemplary hollow fiber membrane 12. Hollow fiber membrane 12 may have a continuous hollow lumen 16 that extends from one end to the other end of the fiber. An outer surface 18 facing outwards forms an outer side of the fiber and an inner surface 20 facing towards the hollow lumen 16 define a fiber portion 26 having a wall thickness 28. The fiber portion 26 is typically porous and contains a polymeric blend comprising a mixture of POLY-1 and POLY-2.

The hollow fiber membranes formed are typically an integrally asymmetric, permeable hollow fiber membrane. As used herein, the term “asymmetric” means that the average pore size varies throughout the wall thickness 28. The term “integrally” means that the pore size changes gradually in size over the wall thickness 28 of the fiber portion 26.

The wall thickness 28 of the fiber portion 26, measured between the outer surface 18 and the inner surface 20 of the fiber 26 portion of the hollow fiber membrane 12, can be in the range of from 10 to 400 micrometers or 20 to 300 micrometers. The fiber portion 26 is formed from the casting solution. The wall thickness can be at least 10, at least 20, at least 30, at least 40, at least 50, at least 60, at least 80, or at least 100 micrometers and up to 400, up to 350, up to 300, up to 275, up to 250, up to 225, up to 200, up to 175, up to 150, up to 125, up to 100, up to 80, or up to 60, or up to 50 micrometers. In some hollow fiber membranes, the wall thickness is in a range of 30 to 200, 30 to 100, 40 to 150, 40 to 100, 40 to 80, or 40 to 60 micrometers.

Similarly, to achieve a desirable flow and/or pressure drop through the lumen of the hollow fiber membranes, the inside diameter of the hollow fiber membranes, which corresponds to the diameter of the inner wall 20 in FIG. 1, is often in the range of from 50 to 800 micrometers.

This diameter can be at least 50, at least 60, at least 80, at least 100, at least 150, at least 200, at least 250, or at least 300 micrometers and up to 800, up to 700, up to 600, up to 500, up to 400, up to 300, or up to 200 micrometers. In some hollow fiber membranes, the inside diameter is in a range of 50 to 700, 50 to 600, 100 to 500, 100 to 400, 100 to 300, or 100 to 200 micrometers.

Wall thicknesses and diameters (i.e., inner or lumen diameter, and outer diameter) of the membranes can be determined using scanning or transmission electron micrographs (SEM or TEM, respectively), for example with a magnification up to 20,000 times. In some embodiments, the hollow fiber membrane can have tortuous structures extending from the inner surface toward the outer surface.

FIG. 2 illustrates a cross-section view of an exemplary hollow fiber membrane 112. Hollow fiber membrane 112 may have a continuous hollow lumen 116, which extends from one end to the other end of the fiber; an outer surface 118 facing outwards, which forms an outer side of the fiber; an inner surface 120 facing towards the hollow lumen 116, which defines the limits of the continuous hollow lumen 116. The fiber portion 126 of the membrane has a cross sectional thickness 128 that begins at the inner surface 120 and extends to the outer surface 118. The pore size progressively changes across the cross-sectional thickness 128. Although the pore size can either progressively increase or decrease across the cross-section zone 128, the pore size often progressively decreases. Across the cross-section zone, for example, the pore size (diameter) often varies in a range from 10 to 100 nanometers but there can be pores having a larger diameter such as up to 500, up to 1000, up to 5000, or even up to 10,000 nanometers.

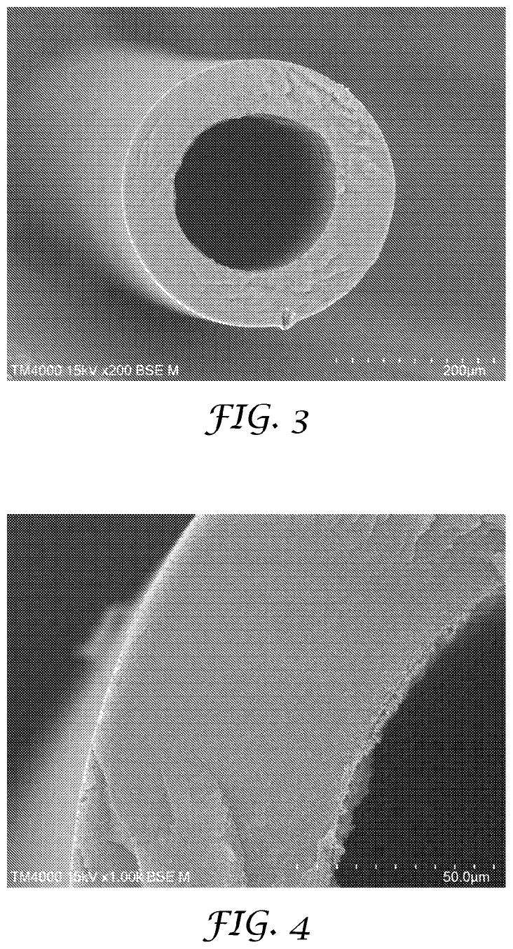

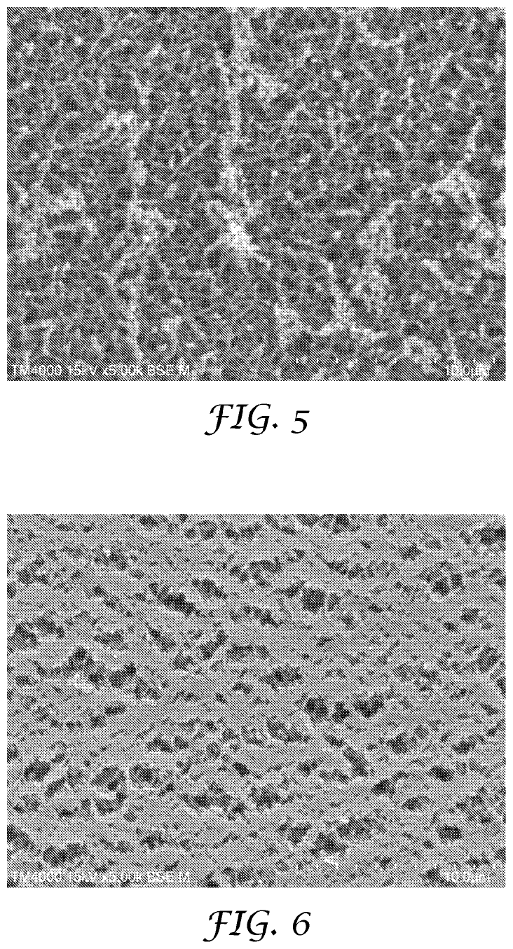

FIG. 3, FIG. 4, FIG. 5, and FIG. 6 are scanning electron micrographs of an exemplary hollow fiber membrane that was formed in Example HFM1 as described below. FIG. 3 and FIG. 4 show a cross section of the hollow fiber membrane at different magnifications, FIG. 5 shows the lumen wall of the hollow fiber membrane, and FIG. 6 shows the outside wall of the hollow fiber membrane. The hollow fiber membrane is porous across its width, but the size and shape of the pores vary across the diameter of the membrane.

The membranes described herein, which are typically prepared using POLY-1 and POLY-2, are advantageous compared to previously known membranes. POLY-1, as described above, is a copolymer that is often prepared by reacting a first macromer of Formula (III) having grafted polymeric sidechains of formula R¹.

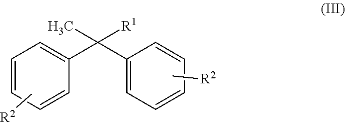

with a second monomer of Formula (IV)

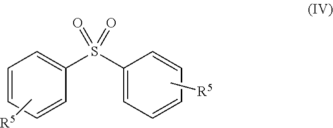

having two functional groups R⁵ that are either nucleophilic groups or leaving groups. The reaction product is typically a copolymer with repeat units connected via an —O— group. Thus, the copolymer has a plurality of groups R¹, which are grafted polymeric sidechains.

Unlike previously prepared membranes, the structural features of POLY-1 can be modified and controlled. For example, both the molecular weight and graft density of the polymeric sidechains can be controlled via stoichiometry during synthesis. Further, the amount of the first macromer used to form POLY-1 can be controlled. This contrasts with other methods where the grafting reaction is performed on a previously formed polymeric material. Thus, the polymeric structure of POLY-1 can be systematically refined to impart desirable characteristics to the membrane such as a particular porosity, pore size, and/or surface hydrophilicity. For example, with respect to fouling resistance versus various bio-foulants (e.g., proteins), detergents, oils, and the like, it is advantageous to optimize the graft density and grafted chain length of the hydrophilic side chains of the copolymer. Resistance to fouling by some foulants may be optimized with short, densely spaced hydrophilic side chains, while resistance to other foulants might best be accomplished with long, more sparsely spaced side chains. With the methodology described herein, POLY-1 is purified by conventional synthetic techniques and provided as a solid for direct incorporation into the membrane casting formulations along with POLY-2 and optionally POLY-3.

Membranes containing POLY-1 provide enhanced fouling resistance compared to membranes lacking POLY-1. For instance, membranes containing POLY-1 do not exhibit a decrease in flux as throughput increases when the feed stream contains a surfactant such as TWEEN-80 as can be seen in FIG. 2. Surfactants, sometimes also known as detergents, are commonly included in solutions containing biomolecules to increase their stability during purification and storage. Therefore, membranes containing POLY-1 are suitable for purification of biomolecules stabilized with surfactants which can increase yield and purity of biomolecules purified using membranes containing POLY-1. Membranes containing POLY-1 may also display enhanced resistance to fouling by biomaterials such as proteins, oils, lipids, or other hydrophobic, amphiphilic, or ionic fluid components.

### Methods of Using the Porous Articles

The porous articles can be used for separating various compositions based on the size of components in the composition. In many embodiments, the porous articles are membranes suitable for separating components of a mixture based on the size of the components. For example, a mixture of biomaterials can be separated based on the size of the various biomaterials in the mixture. The larger biomaterials are typically retained upstream of the membranes and/or pass through (i.e., permeate) the membrane at a slower speed than smaller biomaterials. Thus, the composition exiting the membrane tends to have an enriched concentration of the smaller biomaterials compared to the original mixture of biomaterials.

Thus, a method of separating components based on size is provided. The method includes providing a porous separation article as described above, which is typically a membrane. The method further includes passing a first composition through the membrane, wherein the first composition of biomaterials comprises a plurality of different biomaterials with different average sizes. The method further includes separating the plurality of different biomaterials based on their different average sizes. A first biomaterial that has a smaller average size than a second biomaterial can typically permeate through the membrane more rapidly than the second biomaterial. Likewise, the second biomaterial that has a larger average size than the first biomaterial will be retained by the membrane and/or will permeate through the membrane more slowly than the first biomaterial. Thus, a second composition exiting the membrane will have a higher concentration of the first biomaterial and a lower concentration of the second component compared to the original first composition of biomaterials.

The biomaterials that can be separated include, for example, bacteria, viruses, viral particles, proteins, protein fragments, fusion proteins, cells, cell debris, DNA, RNA, and the like. Different size biomaterials within the same general category as well as biomaterials within different general categories can be separated based on their different average sizes. The second composition exiting the membrane will have a higher concentration of the smaller biomaterials.

For example, the membrane can selectively retain viruses or viral particles having a larger average size than other biomaterials in the first composition of biomaterials. Further different size viruses and/or viral particles can be separated from each other with the smaller viruses and/or viral particles permeating though the membrane more quickly. In a specific example, an adeno-associated virus or a portion of an adeno-associated virus can be separated from larger viruses.

Likewise, different bacteria can be separated based on their average sizes with the smaller bacteria permeating through the membrane more quickly or bacteria can be separated from different types of biomaterials based on average size differences.

Similarly, proteins or protein fragments can be separated from other biomaterials. In some examples, the proteins or protein fragments are smaller than the other biomaterials and can permeate faster though the membrane. The protein or protein fragment can be, for example, a monoclonal antibody, a monoclonal antibody fragment, or a fusion protein.

In some embodiments, the hollow fiber membrane of the present discourse can be used for multiple extracorporeal blood purification procedures including dialysis, blood oxygenation, and plasmapheresis.

### EXAMPLES

Unless otherwise noted or readily apparent from the context, all parts, percentages, ratios, etc. in the Examples and the rest of the specification are by weight.

TABLE 1
Materials Used in the Examples

| | Abbreviation or | |
|---|---|---|
| Reagent | Trade Name | Source |
| N-methylpyrrolidone | NMP | Alfa Aesar |
| 4,4-Bis(4-hydroxyphenyl)pentanoic | Diphenolic Acid | Tokyo Chemical |
| acid | | Industry |
| Bis(4-fluorophenyl) sulfone | — | Oakwood Chemical |
| 4,4′ -Sulfonyldiphenol | — | Oakwood Chemical |
| Poly(ethylene glycol) monomethyl | mPEG 550 | Fluka |
| ether 550 | | |
| Poly(ethylene glycol) monomethyl | mPEG 2000 | Sigma-Aldrich |
| ether 2000 | | |
| Polyethersulfone | PES, available as | BASF Corp, Florham |
| | ULTRASON E 6020 P | Park, NJ |
| Polyethyloxazoline | PEtOx, available | Polymer Chemistry |
| | as AQUAZOL 50 | Innovations, Tucson, AZ |
| Poly(ethylene glycol) 200 g/mol | PEG200 | Thermo Fisher Scientific, |
| | | Fair Lawn, NJ |
| Potassium carbonate | K₂CO₃ | EMD Chemicals |
| Toluene sulfonic acid monohydrate | pTSA | Fisher Scientific |
| Methyl tert-butyl ether for HPLC, | MTBE | Supelco |
| spectrophotometry, and gas | | |
| chromatography | | |
| Toluene | PhCH₃ | Sigma Aldrich |
| Chloroform-d | CDCl₃ | Cambridge Isotope |
| | | Laboratories |
| Dimethylsulfoxide-d₆ | DMSO-d₆ | Cambridge Isotope |
| | | Laboratories |
| Potassium carbonate | K₂CO₃ | Alfa Aesar, Oakwood |
| Lithium bromide | LiBr | Sigma Aldrich |
| Polysorbate 80 | TWEEN-80 | VWR |
| Triethylamine | Et₃N | EMD Millipore |
| Methanesulfonyl chloride | MsCl | Tokyo Chemical Industry |
| N,N-dimethylaminopyridine | DMAP | Alfa Aesar |
| Sodium Azide | NaN₃ | Alfa Aesar |
| Poly(ethylene glycol) monomethyl | mPEG 550 | Tokyo Chemical Industry |
| ether 550 | | (catalog number P2184) |
| 10% Palladium on Carbon | 10% Pd/C | Sigma-Aldrich |
| 5M sodium methoxide in methanol | — | Tokyo Chemical Industry |

### Test Methods

### GPC Method

Gel permeation chromatography data was obtained using an Agilent 1260 Infinity GPC equipped with isocratic pump, standard degasser, standard autosampler, thermostated column compartment set to 55° C., and a refractive index detector using two Agilent PLGel 5 μm Mixed-D 300×7.5 mm gel permeation chromatography columns. Gel permeation chromatography samples were prepared at 5 mg/mL in a solution of 20 mM lithium bromide in N-methylpyrrolidone and then passed over a Pall 0.2 micrometer polypropylene syringe filter. The columns were held at 55° C. and the flow rate was 0.8 m/min. All molecular weight data are reported against Agilent polystyrene standards (Polystyrene calibration kit S-M-10 part #PL2010-0100).

### NMR Spectroscopy Method: Structure Characterization

Nuclear magnetic resonance spectra were obtained using a Bruker Avance III 300 MHz instrument equipped with a room temperature broadband probe or a Bruker Avance III 500 MHz equipped with a broadband cryoprobe. Proton (¹H) spectra were obtained at a 150 pulse angle and a relaxation delay of 4 seconds.

### Scanning Electron Microscopy (SEM)

The membrane pore structure was examined using a scanning electron microscope (obtained under the trade designation “HITACHI TM4000Plus” from NCI Inc., Brooklyn Park, MN). Cross sections were prepared by freeze fracturing samples under liquid nitrogen. A thin layer of gold was sputter coated on the samples to make them conductive. An accelerating voltage of 15 kV, 51.7 μA current, and a working distance of 6-7 mm were used. Images were obtained using backscattered electrons (BSE mode).

### Polymer Examples (i.e., Macromers)

### Polymer Example 1 (PE1): MPEG 550 Diphenolic Acid Ester (n=10)

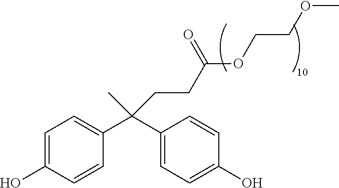

NMR analysis of the poly(ethylene glycol) monomethyl ether 550 used in this experiment had about 10 ethylene glycol units.

A 1 L round bottom flask was charged with a solution of 49.6 g of mPEG 550 dissolved in 400 mL of toluene followed by the addition of 30.1 g of diphenolic acid and 2 g of pTSA monohydrate. The reaction flask was equipped with a Dean-Stark trap and a reflux condenser. The mixture was stirred under an atmosphere of nitrogen and heated to reflux overnight. The water/toluene mixture in the Dean-Stark trap was drained and heating was continued. Three more portions of the toluene distillate were then drained. The reaction mixture was cooled and diluted with ethyl acetate. The mixture was washed with saturated NaHCO₃ solution. The organic portion was washed with brine, dried over Na₂SO₄, filtered, and concentrated under reduced pressure. The resulting syrup was partitioned between CH₂Cl₂ and of 5% Na₂CO₃ solution. The layers were separated, and the organic portion was sequentially washed with water and brine. The organic portion was dried over Na₂SO₄, filtered, and concentrated under reduced pressure to give 67.9 g mPEG 550 diphenolic acid ester as a purple syrup.

¹H NMR (500 MHz, CHLOROFORM-d) δ ppm 7.09-7.25 (m, 2H) 6.98 (d, J=8.6 Hz, 4H) 6.72 (br d, J=8.4 Hz, 4H) 4.07-4.19 (m, 2H) 3.47-3.70 (m, 38H) 3.35 (s, 3H) 2.29-2.41 (m, 2H) 2.06-2.20 (m, 2H) 1.51 (s, 3H).

### Polymer Example 2 (PE2): MPEG 2000 Diphenolic Acid Ester (n=48)

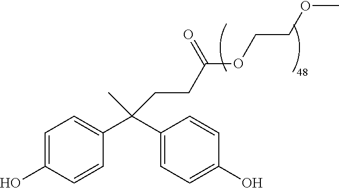

NMR analysis of the poly(ethylene glycol) monomethyl ether 2000 used in this experiment had about 48 ethylene glycol units

To a 3 L 3-neck round bottom flask equipped with a stir bar and Dean-Stark apparatus was added 237.33 g of mPEG 2000, 41.73 g of diphenolic acid, 2.31 g of pTSA monohydrate, and the mixture was suspended in 1.05 kg toluene. The reactant solution was heated to reflux and maintained at reflux for 16 hours. The Dean-Stark apparatus was opened to allow toluene to distill off from the reaction mixture and 902 g of toluene was collected. The crude concentrate was diluted with 500 mL of acetone and poured into 6.2 L of methyl tert-butyl ether. The resulting white precipitate was allowed to settle over 16 hours and the methyl tert-butyl ether was decanted. The precipitate was collected and dried on a countertop in air over the course of 3 days to give 247.86 g of mPEG 2000 diphenolic acid ester as a white solid.

¹H NMR (300 MHz, DMSO-d₆) δ ppm 9.19 (br s, 2H) 6.94 (d, J=8.8 Hz, 4H) 6.66 (d, J=8.4 Hz, 4H) 4.07 (m, 2H) 3.51 (br m, 202H) 3.25 (s, 3H) 2.26 (m, 2H) 2.03 (m, 2H) 1.47 (s, 3H).

### Polymer Example 3 (PE3): MPEG 550 Diphenolic Acid Amide (n 12)

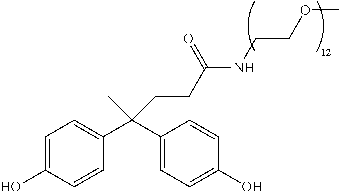

NMR analysis of the poly(ethylene glycol) monomethyl ether 550 (Tokyo Chemical Industry catalog number P2184) used in this experiment had about 12 ethylene glycol units.

A 2 L round bottom flask was charged with a solution of 88.4 g of mPEG 550 dissolved in 500 mL of CH₂Cl₂. Triethylamine (53.7 mL) and DMAP (980 mg) were added to the reaction and the mixture was cooled to 0° C. under an atmosphere of N₂. Methanesulfonyl chloride (14.9 mL) was then added to the stirred mixture over a 10 min period. After stirring overnight, the reaction mixture was quenched by addition on 300 mL of saturated NaHCO₃ solution. The layers were separated, and the aqueous portion was extracted with an additional 75 mL of CH₂Cl₂. The combined organic layers were washed with H₂O (200 mL) and brine (100 mL), dried over Na₂SO₄ and the concentrated under reduced pressure to give 100 g of mPEG 550 methanesulfonate as an orange oil. ¹H NMR (500 MHz, CHLOROFORM-d) δ ppm 3.09 (s, 3H) 3.39 (s, 3H) 3.56 (m, 2H) 3.60-3.70 (m, 42H) 3.76 (m, 2H) 4.39 (m, 2H).

A 500 mL round bottom flask was charged with a solution of 100 g of mPEG 550 methanesulfonate dissolved in 100 mL of anhydrous DMF. Sodium azide (12.2 g) was added, and the stirred mixture was heated to 50° C. under an atmosphere of N₂. After 3 days, the reaction was concentrated under reduced pressure. The resulting syrup was partitioned between 400 mL of CH₂Cl₂ and 400 mL of H₂O. The layers were separated, and the aqueous portion was extracted with an additional 200 mL of CH₂Cl₂. The combined organic layers were washed with H₂O (2×300 mL), dried over Na₂SO₄ and the concentrated under reduced pressure to give 90.4 g of mPEG 550 azide as an amber syrup. ¹H NMR (500 MHz, CHLOROFORM-d) δ ppm 3.35-3.37 (m, 5H) 3.54 (m, 2H) 3.60-3.71 (m, 44H).

A solution of mPEG 550 azide (14.4 g) dissolved in 100 mL of methanol was transferred to a 500 mL Parr bottle. 10% Pd/C (228 mg) was added to the bottle and the mixture was shaken under an atmosphere of H₂ at 50 PSI for 2 h. The reaction mixture was filtered through a pad of Celite and concentrated to give mPEG 550 amine (13.3 g) as a light-colored oil. ¹H NMR (500 MHz, CHLOROFORM-d) δ ppm 2.84 (m, 2H) 3.37 (s, 3H) 3.50 (m, 2H) 3.54 (m, 2H) 3.62 (m, 42H).

mPEG 550 amine (13.3 g) and 4,4-bis(4-hydroxyphenyl)pentanoate (7.14 g, prepared by the method of Sane et al. European Polymer Journal 47 (2011) 1621-1629) were combined in a 500 mL round bottom flask. A 5M solution of sodium methoxide (11.9 mL) was added to the flask and the stirred mixture was heated to 70° C. under an atmosphere of N₂. After stirring for 24 h, the temperature was raised to 85° C. and stirring was continued for 48 h. The reaction mixture was cooled and treated with 1N aqueous HCl solution (60 mL) and 110 mL of 10% MeOH/CHCl₃. The pH of the aqueous phase was adjusted to pH 7 by addition of 1N aqueous NaOH solution. The layers were separated, and the organic portion was dried over Na₂SO₄ and the concentrated under reduced pressure to give a syrup which was treated with toluene and concentrated under reduced pressure to give 19.8 g of mPEG 550 diphenolic acid amide as a syrup. ¹H NMR (500 MHz, CHLOROFORM-d) δ ppm 1.49-1.57 (m, 3H) 1.91-2.01 (m, 2H) 2.35-2.45 (m, 2H) 3.31-3.40 (m, 5H) 3.47-3.51 (m, 2H) 3.51-3.55 (m, 2H) 3.55-3.66 (m, 42H) 6.03 (m, 1H) 6.64-6.75 (m, 4H) 6.90-7.07 (m, 4H).

### Graft Copolymer Examples

### Graft Copolymer Example 1 (GCE1): MPEG 550 Diphenolic Acid/PES Graft Copolymer

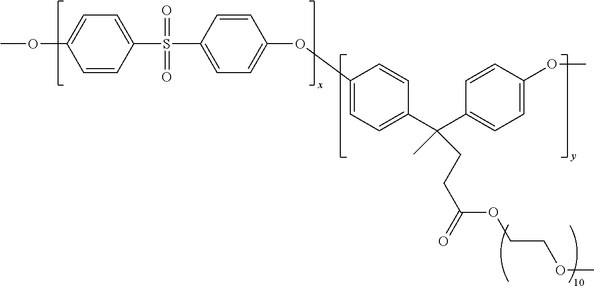

A solution of 67.0 g of mPEG 550 diphenolic acid ester (PE1) dissolved in 400 mL of NMP was added to a 3 L 3-neck round bottom flask. The reaction flask was equipped with a Dean-Stark trap and a reflux condenser. 22.6 g 4,4′-sulfonyl diphenol (90.4 mmol) was added to the reaction mixture along with 50 mL of toluene. The mixture was stirred under an atmosphere of nitrogen and heated to 150° C. After 2 hours, the toluene collected in the Dean-Stark trap was removed and an additional 25 mL of toluene was added to the reaction mixture. Heating was continued and the toluene collected in the Dean-Stark trap was removed. Bis(4-fluorophenyl) sulfone (46.0 g, 180.8 mmol) and K₂CO₃ (29.9 g, 217 mmol) was added to the reaction mixture along with 25 mL of toluene. Heating was continued at 150° C. for 90 min. The temperature was lowered to 110° C. and the mixture continued stirring overnight. The reaction mixture was cooled to about 80° C. and slowly poured into 4 L of ice-cold deionized water with stirring. A precipitated polymer was isolated by filtration through a Buchner funnel and rinsed with water. The collected polymer was stirred with 2 L of deionized water and again isolated by filtration through a Buchner funnel. Washing with deionized water was repeated two more times. The wet polymer was transferred to a 2 L flask and placed under high vacuum at 50° C. until most of the water was removed. The resulting solid was placed in a crystallizing dish and allowed to dry to constant weight to give 105 g mPEG 550 diphenolic acid/PES copolymer (GCE1) as a light tan solid. GPC analysis showed that the Mn was 13,103 g/mol, Mw was 48,570 g/mol, and PDI was 3.71.

### Graft Copolymer Example 2 (GCE2): MPEG 550 Diphenolic Acid/PES Graft Copolymer

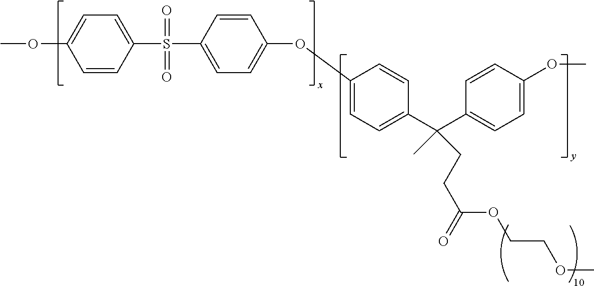

To a 3 L resin kettle was added 173.43 g of 4,4′-sulfonyldiphenol (693 mmol), 234.93 g, of bis(4-fluorophenyl) sulfone (924 mmol), 306.49 g of K₂CO₃ (2.22 mol), 200.37 g of mPEG 550 diphenolic acid ester (PE1) and 924 g NMP. The resin kettle was equipped with a heating mantle, J-KEM temperature controller with a 0.25 inch stainless steel thermocouple, a mechanical stirrer equipped with a stainless-steel stir shaft utilizing a Teflon stir blade backed by a stainless steel blade and 4-prong propeller, and a 0.25 inch stainless steel dip tube connected to a Schlenk line for N₂ sparging, and a short path distillation head with a 1 L receiving flask cooled in a dry ice acetone bath. To the resin kettle was added 346 g of toluene and the reaction mixture was heated to 150° C. for 2 hours to distill off toluene, the short path distillation head was exchanged for a glass stopper, the dip tube was placed above the reactant solution, and the temperature increased to 165° C. The reaction mixture was maintained at 165° C. for 3 hours and 15 minutes before cooling to room temperature and the reaction mixture was allowed to stand at room temperature for 18 hours. The reaction mixture was diluted with 300 mL N,N-dimethylformamide and poured into 12 L water to precipitate the polymer. The polymer was blended in an industrial blender with water and collected by filtration. The filtered product was then soaked in 12 L water at 40° C. with mechanical stirring and the water exchanged after 3 hours, the polymer soaked at 40° C. for a further 3 hours, the water exchanged once more, and the polymer was soaked at 25° C. for 16 hours before collecting by filtration. The polymer was dried under an N₂ stream in a pressure funnel for 6 days to give 473.67 g of mPEG 550 diphenolic acid/PES copolymer (GCE2). GPC analysis showed that the PDI was 6.38, Mn was 30,317 g/mol, and Mw was 193,549 g/mol.

### Graft Copolymer Example 3 (GCE3): MPEG 2000 Diphenolic Acid/PES Graft Copolymer

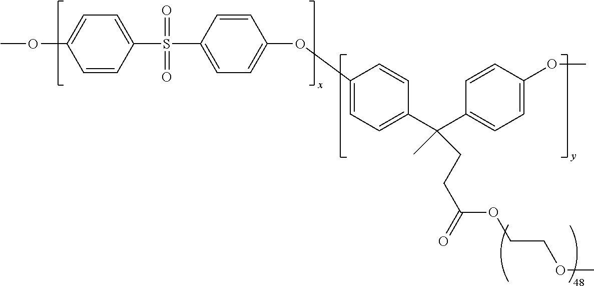

To a 3 L resin kettle was added 73.97 of g 4,4′-sulfonyldiphenol (296 mmol), 100.23 g of bis(4-fluorophenyl) sulfone (394 mmol), 108.96 g of K₂CO₃ (1.58 mol), 240.37 g of mPEG 2000 diphenolic acid ester (PE2), and 700 g of NMP. The resin kettle was equipped with a heating mantle, J-KEM temperature controller with a ¼″ stainless steel thermocouple, a mechanical stirrer equipped with a stainless-steel stir shaft utilizing a Teflon stir blade backed by a stainless steel blade and 4-prong propeller, and a 0.25 inch stainless steel dip tube connected to a Schlenk line for N₂ sparging, and a short path distillation head with a 1 L receiving flask cooled in a dry ice acetone bath. To the resin kettle was added 262 g of toluene and the reaction mixture was heated to 150° C. for 1.5 hours to distill off toluene. Once the toluene had been distilled, the short path distillation head was exchanged for a glass stopper, the dip tube was placed above the reactant solution, and the reaction was maintained at 150° C. for 6 hours and 25 minutes before cooling to room temperature and allowing the reactant solution to stand at room temperature for 11.5 hours. The reaction mixture was poured into 12 L water and the precipitate was cut into small approximately 1×1 cm chunks with scissors. The material was then soaked in 12 L water at room temperature for 4 hours, the water exchanged, and the process repeated for a total of 4 iterations. GPC analysis showed that the PDI was 2.48, Mn was 58,572 g/mol, and Mw was 145,272 g/mol.

### Graft Copolymer Example 4 (GCE4): MPEG 550 Diphenolic Amide/PES Graft Copolymer

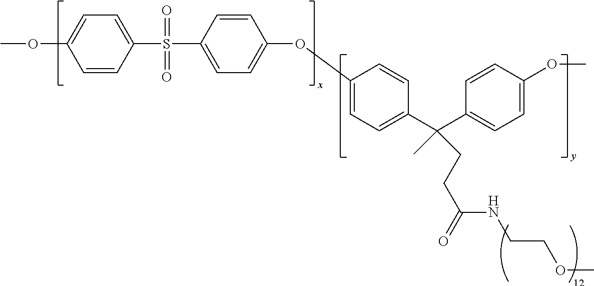

A solution of 19.8 g of mPEG 550 diphenolic acid amide (PE3) dissolved in 100 mL of NMP was added to a 300 mL 3-neck round bottom flask. The reaction flask was equipped with a Dean-Stark trap and a reflux condenser. The mixture was treated with 20 mL of toluene and heated to 150° C. and sparged with a flow of N₂ until all of the toluene was collected in the Dean-Stark trap. The mixture was then cooled to 70° C. and 4,4′-sulfonyl diphenol (6.30 g), bis(4-fluorophenyl) sulfone (12.81 g) and K₂CO₃ (8.41 g) were added to the reaction mixture along with 20 mL of toluene. The temperature was raised to 150° C. until all of the toluene was collected in the Dean-Stark trap. Another 20 mL of toluene was added to the reaction mixture and the temperature was raised temp to 165° C. for 3 h. The temperature was lowered to 110° C. and the mixture continued stirring overnight. The reaction mixture was cooled to about 80° C. and slowly poured into 1 L of ice-cold deionized water with stirring. A precipitated polymer was isolated by filtration through a Buchner funnel and rinsed with water. The collected polymer was stirred with 1 L of deionized water and again isolated by filtration through a Buchner funnel. Washing with deionized water was repeated two more times. The wet polymer was transferred to a 2 L flask and placed under high vacuum at 50° C. until most of the water was removed. The resulting solid was placed in a crystallizing dish and allowed to dry to constant weight to give 33.5 g mPEG 550 diphenolic acid amide/PES copolymer (GCE4) as a light tan solid. GPC analysis showed that the Mn was 24,307 g/mol, Mw was 48,335 g/mol, and PDI was 1.99.

### Preparation of Polymer Dope (i.e., Casting Solution) and Hollow Fiber Membrane

### Hollow Fiber Membrane Example 1 (HFM1): Prepared with GCE1

A polymer dope was prepared with the following composition: 23 weight % PES, 9 weight % PEtOx, 2.6 weight % GCE1, 30.3 weight % PEG200, 33.2 weight % NMP, and 2 weight % deionized water. The polymer dope was mixed using a centrifugal mixer (available as “SPEEDMIXER” from FlackTek, Landrum, SC) for 15 seconds at 800 rpm, followed by 9.75 minutes at 1200 rpm. The polymer dope was transferred to a hopper, heated to 50° C., and was degassed at 200 mbar. The resulting polymer dope was transparent with a brownish tint and was macroscopically homogeneous.

A gear pump (available as “MODEL H-9000” from Zenith Pumps, Monroe, NC) was used to pump the polymer dope from the hopper to a spinneret die with an inner channel for a bore liquid and an annular gap for the polymer dope, separated by a needle. The flow path was heated to 50° C. and included a 15 micrometer in-line filter. The spinneret die had an annular gap of 410 micrometers, a needle outer diameter of 300 micrometers, and a needle inner diameter of 150 micrometers. The spinneret die was fixed at a height of 20 cm above an aqueous precipitation bath and was heated to 50° C. The bore liquid consisted of 50 weight % PEG200, 45 weight % NMP, and 5 weight % deionized water.

The extruded polymer dope fell through a climate-controlled zone with an air temperature of 45° C. and a relative humidity of 96%. Air was blown through the climate-controlled zone to achieve a steam mass flow rate of 2.8 kg/h. The extruded polymer dope then entered an aqueous precipitation bath heated to 50° C., thereby vitrifying the pore structure of the hollow fiber membrane. The hollow fiber membrane was collected at a line speed of 150 ft/min and wound on a drum.

The resulting hollow fiber membrane bundle was flushed down the lumen volume with approximately 4 L of deionized water, then extracted in water heated to 90° C. for 1 hour, and finally dried at room temperature overnight. The hollow fiber membrane had an inner diameter of 203 micrometers and an average wall thickness of 70 micrometers.

Scanning electron micrographs of the hollow fiber membrane of Example HFM1 are in FIG. 3 (cross section at a magnification of 200 times), FIG. 4 (cross section at a magnification of 1,000 times), FIG. 5 (inner lumen wall at a magnification of 5,000 times), and FIG. 6 (outside wall at a magnification of 5,000 times).

### Comparative Hollow Fiber Membrane Example 1 (CHFM1)

A polymer dope was prepared as in HFM1, but with the following composition: 25.5 wt % PES, 9 wt % poly(2-ethyl-2-oxazoline), 30.3 wt % poly(ethylene glycol) with a molecular weight of 200 g/mol, 33.2 wt % NVP, and 2 wt % deionized water. The polymer dope was transferred to a hopper, heated to 50° C., and allowed to degas overnight. The resulting polymer dope was transparent and macroscopically homogeneous.

A gear pump (available as “MODEL H-9000” from Zenith Pumps, Monroe, NC) was used to pump the polymer dope from the hopper to a spinneret die with an inner channel for a bore liquid and an annular gap for the polymer dope, separated by a needle. The flow path was heated to 50° C. and included a 15 micrometer in-line filter. The spinneret die had an annular gap of 410 micrometers, a needle outer diameter of 300 micrometers, and a needle inner diameter of 150 micrometers. The spinneret die was fixed at a height of 20 cm above an aqueous precipitation bath and was heated to 50° C. The bore liquid consisted of 50 weight % PEG200, 45 weight % NMP, and 5 weight % deionized water.

The extruded polymer dope fell through a climate-controlled zone with an air temperature of 45° C. and a relative humidity of 96%. Air was blown through the climate-controlled zone to achieve a steam mass flow rate of 2.8 kg/h. The extruded polymer dope then entered an aqueous precipitation bath heated to 50° C., thereby vitrifying the pore structure of the hollow fiber membrane. The hollow fiber membrane was collected at a line speed of 150 ft/min and wound on a drum.

The resulting hollow fiber membrane bundle was extracted in water heated to 90° C. for 1 hour, then dried at room temperature overnight. The hollow fiber membrane had an inner diameter of 194 micrometers and an average wall thickness of 70 micrometers.

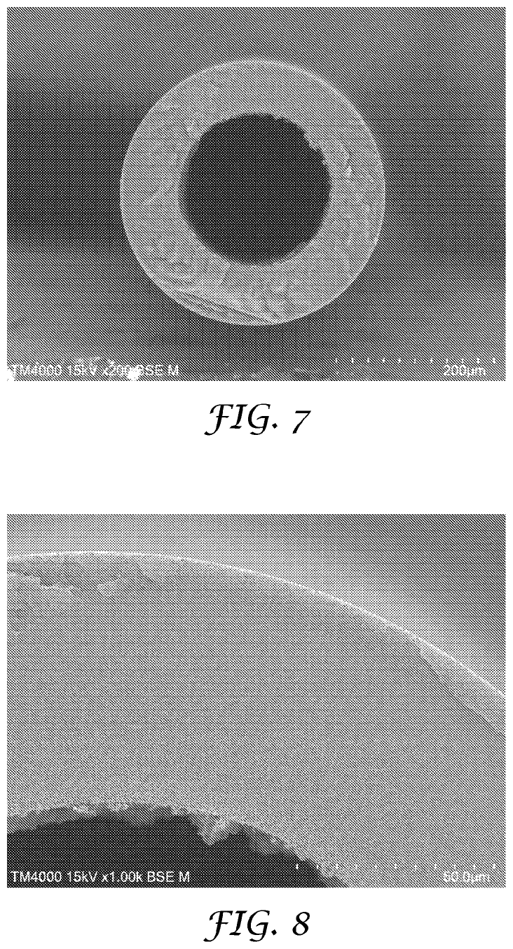

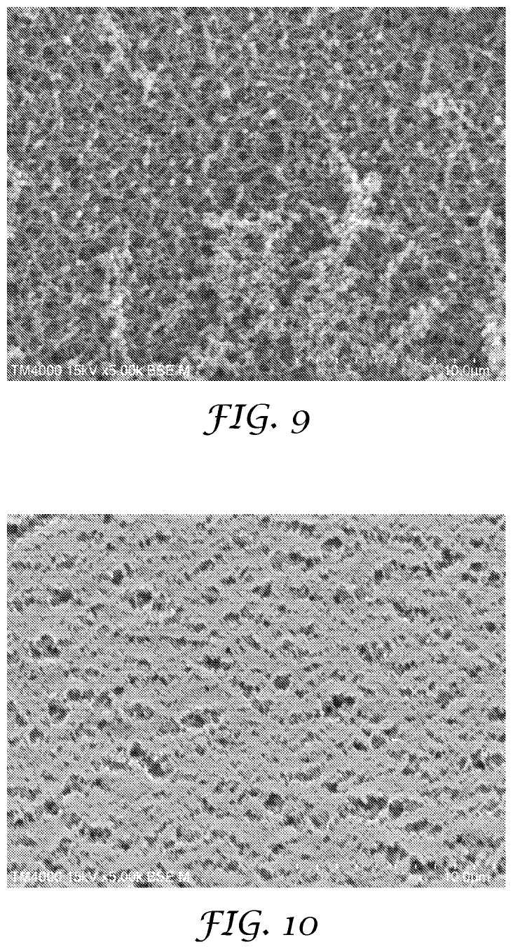

Scanning electron micrographs of the hollow fiber membrane of Comparative Example CHFM1 are in FIG. 7 (cross section at a magnification of 200 times), FIG. 8 (cross section at a magnification of 1,000 times), FIG. 9 (inner lumen wall at a magnification of 5,000 times), and FIG. 10 (outside wall at a magnification of 5,000 times).

### Fouling Resistance Measurement of Hollow Fiber Membranes HFM1 and CHFM1

Hollow fiber membrane modules were made containing the hollow fiber membranes of Example HFM1 and Comparative Example CHFM1. To make each module, two 2.5-inch sections of 0.25 inch nylon tubing (Part No. 2VDL8, Grainger) were cut. The tubing sections were inserted into opposite ends of a polypropylene push-connect T-fitting (Part No. PP0208W-US, John Guest). Three membrane fibers of either Example HFM1 or Comparative Example CHFM1, each longer than the assembly described above, were passed through the resulting assembly from the open end of one section of nylon tubing to the open end of the opposite section. The open end of each section of nylon tubing was then potted with a roughly 0.25 inch plug of epoxy adhesive (LOCTITE EA608, Henkel). The epoxy adhesive was allowed to cure for approximately 20 minutes, after which the plug of epoxy adhesive at each end of the assembly was cut at approximately its halfway point with a razor blade to expose the open ends of the hollow fibers. Each module had a feed inlet at one potted end, a feed outlet at the opposite potted end, and a permeate outlet at the open connection of the T-fitting. The effective filtration area of each module was calculated as the surface area of three cylinders having the length of the membrane module and a diameter equal to the inner diameter of the hollow fiber membrane and had a value of about 2 cm².

PBS buffer solution was prepared by dissolving 1 packet of PBS buffer powder per liter of deionized water. 1 g of TWEEN-80 was added to each liter of a portion of the PBS buffer solution to make a 0.1 weight percent TWEEN-80 solution in PBS buffer. TWEEN-80 is a nonionic detergent, present in some biopharmaceutical cell culture fluids, which is known to foul the surfaces of many filtration membranes. Both solutions were sterile filtered (NALGENE RAPID-FLOW Sterile Disposable Bottle Top Filter with 0.2-micron PES Membrane).

Flow rate across a hollow fiber module of each type as a function of volumetric throughput was measured at a constant pressure of 2.07 bar (30 psi) while filtering, first, PBS solution, followed by, second, 0.1 weight percent TWEEN-80 in PBS using a PendoTECH NFF Filter Screening System (PendoTECH, Princeton, NJ). The feed inlet of each membrane module was fluidly connected to a 700-mL pressure vessel by an assembly consisting of a Luer stopcock (Part No. EW-12023-27, Cole-Parmer) connected to the pressure vessel, a barbed Luer connector, a segment of ¼-inch tubing, and a polypropylene push-connect union (Part No. PP0408W-US) connected to the membrane module. The feed outlet of each membrane module was connected to an assembly comprising a polypropylene push-connect union, a segment of ¼-inch tubing, a barbed Luer connected, and a Luer cap enabling the feed outlet to be open or closed. The permeate outlet of each membrane module was positioned above a container on a mass balance of the filter screening system. The filter screening system was connected to a computer running software enabling the automated logging of the mass reading on the mass balances every ten seconds.

The stopcock upstream of each membrane module was initially closed, and the pressure vessel upstream of each membrane module was filled with PBS solution. Each pressure vessel was closed and pressurized to 2.07 bar (30 psi) with a compressed air supply and a pressure regulator. The lumens of each hollow fiber module were then vented by opening the Luer cap at the feed outlet and opening the upstream stopcock until buffer was observed exiting the feed outlet. The permeate side of each module was then vented by closing the Luer cap at the feed outlet and observing buffer exiting the permeate outlet. The stopcock upstream of each membrane module was then closed. Automated collection of mass balance readings was then started and the stopcock upstream of each membrane module was opened to initiate flow of PBS buffer solution through both membrane modules. Flow of buffer was allowed to continue for approximately 10 minutes. The stopcock upstream of each module was then closed to stop flow of buffer. Cumulative volumetric throughput (assuming a buffer density of 1 g/mL) was plotted as a function of time for each membrane module, and a linear relationship was observed in both cases. A regression line was fit to each data set, and the slope of the regression line was recorded as the pure buffer flow rate for each membrane module. The pure buffer flow rate for each module was divided by the effective filtration area of each module (2 cm²) and the upstream pressure (2.07 bar) to obtain the pure buffer flux (J_{PBS}) of each membrane module. The pure buffer fluxes of the membrane modules containing the hollow fiber membranes of Example HFM1 and Comparative Example CHFM1 were 1.51 and 1.85 mL/(cm²-min-bar), respectively.

Each of the pressure vessels was then emptied and re-filled with the PBS buffer containing 0.1 weight percent TWEEN-80 as a membrane foulant. The pressure vessels were closed and re-pressurized to 2.07 bar (30 psi). Automated mass logging was re-initiated, and the stopcocks upstream of the membrane modules were opened to initiate flow of the TWEEN-80 solution, which was allowed to continue for 2.9 hours, after which the stopcocks were closed. Data analysis was then conducted to calculate the membrane flux as a function of volumetric throughput (assuming a fluid density of 1 g/mL) for each membrane module. The flow rate at each time point was calculated as the difference in throughput relative to the prior time point divided by the difference in time relative to the prior time point. At each time point, the flux was then calculated by dividing the flow rate by the effective filtration area and the upstream pressure to obtain the membrane flux. A ratio of the membrane flux to the pure buffer flux (J/J_{PBS}) was calculated for each membrane type at each time point. Finally, the data was smoothed by calculating, at each time point, a running average of the seven J/J_{PBS} values centered on that time point.

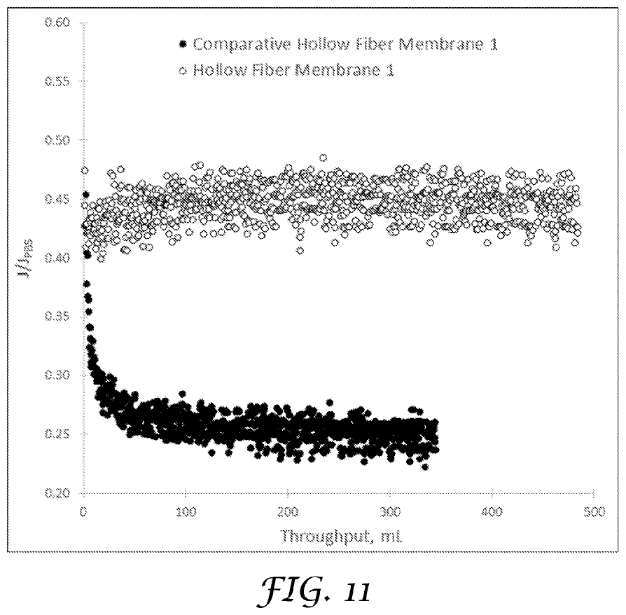

FIG. 11 is a plot of smoothed J/J_{PBS} as a function of volumetric throughput. The initial flux for each membrane was slightly less than half the pure buffer flux, probably due to the higher viscosity of the TWEEN-80 solution. The flux of the membrane of Comparative Example CHFM1 then rapidly decreased during filtration of the TWEEN-80 solution before stabilizing at a lower value of only about 25% of the pure buffer flux. This flux decrease is believed to be due to fouling of the membrane pore structures by adsorption of TWEEN-80. The hollow fiber membrane of Example HFM1 maintained the initial value of its flux in the TWEEN-80 solution, which is believed to be due to reduced adsorption of TWEEN-80 on the membrane of Example HFM1 due to the presence of hydrophilic poly(ethylene glycol) chains of GCE1 present at the membrane pore surfaces. This example demonstrates that improved fouling resistance of membranes can be achieved by incorporation of water-insoluble, amphiphilic sulfone copolymers of this invention.

1. A copolymer comprising a plurality of repeat units joined by —O— groups, wherein the plurality of repeat units comprises:
a) repeat units of Formula (I) 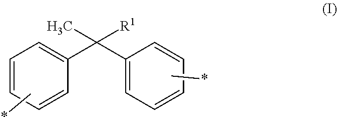
b) repeat units of Formula (II) 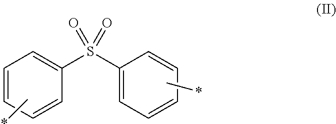
wherein
R¹ comprises a plurality of repeat groups of formula —X—(CH₂)_{y}—;
X is —O— or —NH—; and
each y is an integer in a range of 1 to 4; and
an asterisk (*) is an attachment site to the —O— group that joins two repeat units.

2. The copolymer of claim 1, wherein the repeat units of Formula (I) are of Formula (I-A) 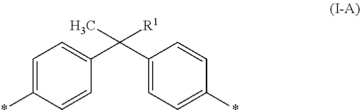
and the repeat units of Formula (II) are of Formula (II-A)

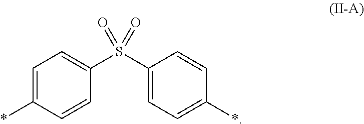

3. The copolymer of claim 1, wherein R¹ is a group of formula
—R³—C(═O)—Z—[(CH₂)_{y}—X]ₙR⁴ wherein
R³ is an alkylene;
R⁴ is a terminal group;
Z is —O— or —NH—;
y in an integer in a range of 1 to 4; and
n is an integer in a range of 3 to 1000.

4. The copolymer of claim 3, wherein R¹ is a group of formula —CH₂CH₂—C(═O)—O—[(CH₂CH₂—O]ₙR⁴ where R⁴ is an alkyl.

5. The copolymer of claim 1, wherein the copolymer comprises 10 to 60 weight percent of the first repeat unit of Formula (I).

6. The copolymer of claim 1, wherein the plurality of repeat units further comprises a third repeat unit that is different than the first repeat unit and the second repeat unit, wherein the third repeat unit is of Formula (V-A), Formula (V-B), Formula (V-C), Formula (V-D), Formula (V-E), Formula (V-F), or a combination thereof 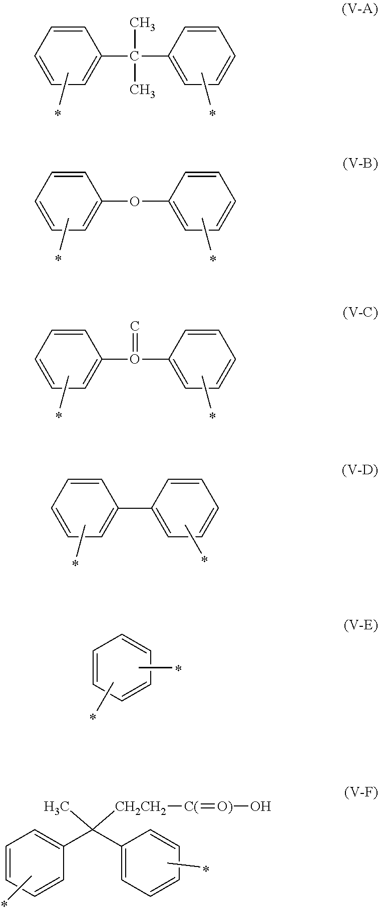
wherein an asterisk (*) is the attachment site to an —O— group that joins repeat units.

7. The copolymer of claim 1, wherein the weight average molecular weight is in a range of 10,000 to 250,000 grams/mole.

8. A porous polymeric article comprising a first copolymer comprising a plurality of repeat units joined by —O— groups, wherein the plurality of repeat units comprises:
a) repeat units of Formula (I) 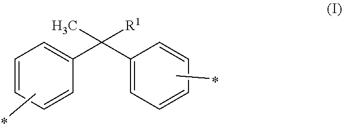
b) repeat units of Formula (II) 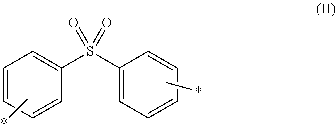
wherein
R¹ comprises a plurality of repeat groups of formula —X—(CH₂)_{y}—;
X is —O— or —NH—; and
each y is an integer in a range of 1 to 4; and
an asterisk (*) is an attachment site to the —O— group that joins two repeat units.

9. The porous polymeric article of claim 8, wherein the porous polymeric article is a membrane comprising 1 to 50 weight percent of the first copolymer and 50 to 99 weight percent of
a second polymer that is an aromatic polyether sulfone having repeat units of Formula (II) 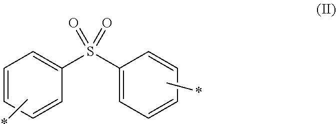
wherein an asterisk (*) is the attachment site to an —O— group that joins two repeat units.

10. The porous polymeric article of claim 9, wherein the second polymer further comprises repeat units of Formula (V-A), Formula (V-B), Formula (V-C), Formula (V-D), Formula (V-E), Formula (V-F), or a mixture thereof

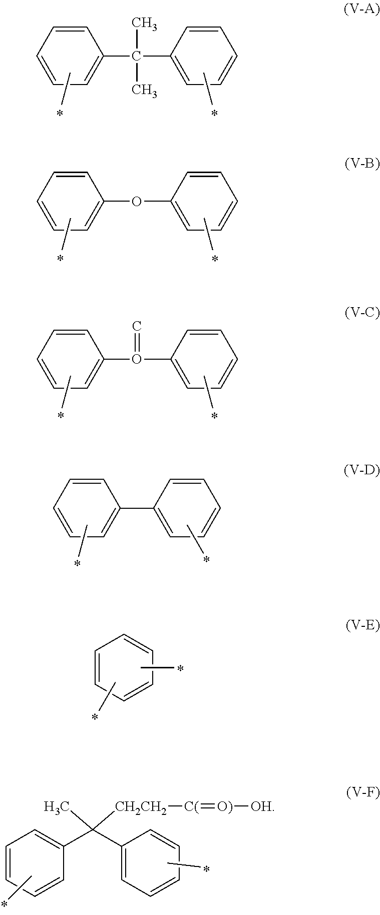

11. The porous polymeric article of claim 8, wherein the membrane is a hollow fiber membrane.

12. A method of separating biomaterials based on size differences, the method comprising:
providing a porous polymeric article comprising a first copolymer comprising a plurality of repeat units joined by —O— groups, wherein the plurality of repeat units comprises:
a) repeat units of Formula (I) 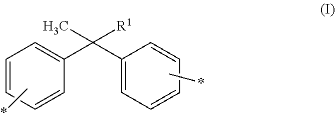
b) repeat units of Formula (II) 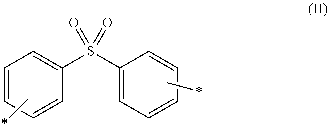
wherein
R¹ comprises a plurality of repeat groups of formula —X—(CH₂)_{y}—;
X is —O— or —NH—; and
each y is an integer in a range of 1 to 4; and
an asterisk (*) is an attachment site to the —O— group that joins two repeat units;
passing an aqueous mixture of biomaterials through the porous polymeric material, wherein the mixture of biomaterials comprises biomaterials having different average sizes; and
separating the mixture of biomaterials based on their average sizes, wherein a first biomaterial that is smaller than a second biomaterial permeates through the porous polymeric article at a faster rate.
